(19) United States
(12) Patent Application Publication
(10) Pub. No.: US 2025/0269063 A2
(43) Pub. Date: Aug. 28, 2025

(54) CODON-OPTIMIZED SMAD7 GENE THERAPY TO TREAT AND PREVENT MUSCLE WASTING AND TO ENHANCE MUSCLE MASS

(71) Applicant: AAVogen Inc., Rockville, MD (US)

(72) Inventor: Buel D. Rodgers, Rockville, MD (US)

(73) Assignee: AAVogen Inc., Rockville, MD (US)

(21) Appl. No.: 18/834,058

(22) PCT Filed: Feb. 6, 2023
(86) PCT No.: PCT/US2023/062030

§ 371 Date: Jul. 29, 2024

Related U.S. Application Data
(60) Provisional application No. 63/307,472, filed on Feb. 7, 2022.

(51) Int. Cl. A61K 48/00; C07K 14/495; C12N 15/86

(52) U.S. Cl. CPC A61K 48/0058; C07K 14/495; C12N 15/86; C12N 2750/14143; C12N 2800/22

(57) ABSTRACT

Provided herein are codon-optimized Smad7 polynucleotides and vectors comprising codon-optimized Smad7 polynucleotides for use in increasing or prolonging Smad7 expression in a subject.

### PRIORITY

This application claims the benefit of U.S. Ser. No. 63/307,472, filed on Feb. 7, 2022, which is incorporated by reference in its entirety herein.

### ACKNOWLEDGEMENT OF FEDERAL FUNDING

This invention was made with government support under contracts R44CA221539 & R43AR075438 awarded by the National Institutes of Health. The Government has certain rights in the invention.

### INCORPORATION BY REFERENCE OF SEQUENCE LISTING

The instant application contains a Sequence Listing which has been submitted in xml format via Patent Center and is hereby incorporated by reference in its entirety. Said xml copy, created on Feb. 6, 2023, is named “430257-991001-Sequence_Listing.xml” and is about 18507 bytes in size.

### BACKGROUND

Significant wasting of striated muscle, which includes skeletal and cardiac muscle but not smooth muscle, occurs in a number of diverse primary indications and diseases (Cohen et al., Nat Rev Drug Discov 14, 58-74, 2015; Bodine and Baehr Am J Physiol Endocrinol Metab 307, E469-484, 2014), many of which are not always obvious. It is inherent to neuromuscular diseases including the muscular dystrophies and myopathies, it occurs in most chronic disease states and it can be disguised in obesity-related disorders. These include sarcopenic obesity and type 2 diabetes mellitus where elevated serum levels of myostatin, tumor necrosis factor (TNF)α, interleukin (IL)-6 and other inflammatory cytokines drive muscle loss in the presence of heightened adiposity (Sakuma and Yamaguchi International journal of endocrinology 2013, 204164, 2013; Biolo et al., Clin Nutr 33, 737-748, 2014; Workeneh and Bajaj Int J Biochem Cell Biol 2013; Wang et al., J Huazhong Univ Sci Technolog Med Sci 32, 534-539, 2012; Amor et al., Exp Clin Endocrinol Diabetes 127, 550-556, 2019; Chung et al., J Diabetes Complications 34, 107592, 2020; Dial et al., Physiol Rep 8, e14500, 2020). Often commercially overlooked is muscle wasting with musculoskeletal injuries (MSIs) and in subjects exposed to prolonged spaceflight/micro-gravity due to reduced use or gravitational load (Demontis et al., Front Physiol 8, 547, 2017; Tanaka et al., J Physiol Sci 67, 271-281, 2017; Bettis et al., Osteoporos Int 29, 1713-1720, 2018). Addressing this problem could have a profound effect on the military as the muscle wasting caused by MSIs, not denervation, is the primary medical problem compromising military readiness (Zambraski and Yancosek J Strength Cond Res 26 Suppl 2, S101-106, 2012; Bell et al., Disabil Health J 1, 14-24, 2008; Corona et al., J Rehabil Res Dev 52, 785-792, 2015; Garg et al., J Orthop Res 33, 40-46, 2015; Rivera and Corona US Army Med Dep J 30-34, 2016). In fact, 90% of denervated muscles become re-innervated within a year, yet only 10% of muscle strength is ever permanently restored (Stefancic et al., Muscle Nerve 2016).

Muscle wasting occurs with cancer due to tumor-derived or-responsive factors that induce cachexia; a state of pronounced weight loss, frailty and fatigue characterized by severe atrophy of striated muscle (skeletal and cardiac) and fat, in up to 80% of patients with advanced cancers (Fearon et al., Cell metabolism 16, 153-166, 2012; Rausch et al., Oncogenesis 10, 1, 2021). It occurs in more than half of people over 80 who suffer from sarcopenia, the age-related progressive loss of muscle that significantly increases risk for hospitalization, disability and mortality (Cruz-Jentoft et al., Curr Opin Clin Nutr Metab Care 13, 1-7). Additional disease indications with muscle wasting include heart failure, chronic obstructive pulmonary disease (COPD), end-stage renal disease, chronic infection, hip fracture, malnutrition and burns and sepsis, which is an inexhausted list (Cohen et al., Nat Rev Drug Discov 14, 58-74, 2015). Common to these conditions is an elevated stress or inflammatory response where production of stress hormones (e.g. cortisol), cytokines (e.g. IL-1 & -6) and myokines (e.g. myostatin) that directly induce muscle wasting are elevated (Zhang et al., Med Hypotheses 69, 310-321, 2007; Schakman et al., Int J Biochem Cell Biol 45, 2163-2172, 2013; Zhou et al., Trends Endocrinol Metab 27, 335-347, 2016). This includes multiple members of the transforming growth factor (TGF)β superfamily of secreted ligands that suppress muscle growth, induce muscle atrophy and antagonize the actions of muscle growth promoters (Rodgers and Ward Endocr Rev 2021).

Much interest has centered on the therapeutic prospects of inhibiting these ligands, especially those that bind and activate the Type II activin receptors (ActRIIA and ActRIIB, a.k.a. ACVR2 and ACVR2B) as this pathway stimulates muscle catabolism and expression of these ligands is often elevated with muscle wasting (Rodgers and Ward Endocr Rev 2021; Sartori et al., Trends Endocrinol Metab 25, 464-471, 2014). These ligands include myostatin, the activins (Act A, B & AB) and growth/differentiation factor (GDF)11, all of which appear to signal primarily through ActRIIB. In fact, circulating forms of modified ActRIIB ligand traps that bind multiple ActRII ligands or immunoneutralizing agents (e.g. monoclonal antibodies or peptibodies) targeting myostatin can reverse muscle wasting and increase lifespan in animal models of muscle disease (Rodgers and Ward Endocr Rev 2021). This occurs despite the fact that other pro-cachectic or atrophy-producing cytokines remain elevated. Thus, interventions that selectively prevent ligand activation of ActRIIB or its intracellular signaling in muscle could prove instrumental in treating a variety of muscle disease indications including those discussed herein (Rodgers and Ward Endocr Rev 2021). Targeting ActRIIB ligands in circulating or extracellular environments, however, can produce adverse and serious off-target effects as these ligands are critical to many organ systems including reproduction and angiogenesis.

For instance, a clinical study of ACE-031, a peptibody ligand trap largely composed of the ActRIIB extracellular domain, was terminated prematurely (Campbell et al., Muscle Nerve 55, 458-464, 2017) due to the induction of signs often seen in patients with heredity hemorrhagic telangiectasia (HHT), which included bleeding from mucous membranes and skin vasodilation. This disease results from mutations in two signaling proteins, endoglin or activin like kinase-1, that ultimately impair TGFβ receptor signaling in endothelial cells. This compromises blood vessel integrity in humans and mouse models of the disease and can cause hemorrhaging in various tissues. In mice, the nose, ears, tail and in several internal organs are affected, especially the liver and lungs, while some mice even suffer strokes from arteriovenous malformations in the brain (Cunha et al., Circ Res 121, 981-999, 2017; Tual-Chalot et al., Front Genet 6, 25, 2015).

Authors of the ACE-031 study suggest that bone morphogenic protein (BMP)9 attenuation was likely the cause as BMP9 maintains endothelial cells, stimulates angiogenesis and signals via ALK-1, although BMP10 and even GDF11 have similar actions (Tillet and Bailly Front Genet 5, 456, 2014; Zhang et al., Journal of cellular and molecular medicine 24, 8703-8717, 2020). In fact, the developers tested a structurally similar ligand trap, ActRIIa-Fc, and discovered that it indeed attenuates GDF11, myostatin and activin in endothelial cells (Yung et al., Science translational medicine 12, 2020). It also blocks arteriolar remodeling while stimulating vascular apoptosis in animal models of pulmonary hypertension. These surprising results suggest that GDF11, myostatin and activin participate in blood vessel maintenance and in the pathogenesis of pulmonary hypertension. They also raise safety concerns for any ActRIIb-attenuator that functions via ligand sequestration. This obviously includes ligand traps that recognize multiple ActRIIA and ActRIIB ligands, but also drugs that target specific ligands (e.g., myostatin, GDF11, ActA, etc.) for these receptors.

Once activated, ActRIIB recruits Type I activin receptors (activin like kinase (ALK)4/5) to form an activated ActRIIB:ALK4/5 complex that phosphorylates Smad2/3 (Rodgers and Ward Endocr Rev 2021). These receptor Smads then bind Smad4, allowing the complex to enter the nucleus and modify a protein degradation transcriptional program that up-regulates the E3 ubiquitin ligases, MuRF1 and MAFbx, and dephosphorylates Akt. This pathway also upregulates Smad7 expression as a form of intracellular negative feedback, which prevents Smad2/3 phosphorylation and Smad2/3-Smad4 complex formation while promoting degradation of the ActRIIB:ALK4/5 receptor complex.

### SUMMARY OF EXEMPLARY ASPECTS

Because the efficacy of gene therapeutics in general are dependent upon the protein expression level of the encoded transgene, codon-optimization can be used to improve translational efficiency and thus, gene therapy efficacy. This is based on the fact that synonymous changes in the coding sequence, those that alter mRNA but not corresponding amino acid sequences, can substantially improve protein expression levels. Increasing and/or prolonging protein expression levels with codon optimization can also reduce the need to administer high viral titers, which in turn reduces manufacturing costs and can help eliminate potential off-target or immune-related toxicities. This is particularly important for gene therapeutics administered systemically as manufacturing capacity is often a limiting factor.

For these reasons, we optimized the human Smad7 cDNA sequence based on the human codon usage bias. Three independent algorithms were performed and a resulting composite sequence was generated according to ideal characteristics. These changes improved the codon adaptation index (CAI) significantly and removed several motifs capable of causing secondary structure problems that reduce translation efficiency. The latter include a direct repeat (GGAGGAGGAGGA (SEQ ID NO: 8) starting at positions 91 and 151), several hairpins and antiviral motifs. Three negative cis elements were also removed including a splice cite (587ATCACC592), a polyT site (651TTTT654) and a polyA site (946AAAA949). We additionally demonstrated this optimized cDNA sequence to improve muscle Smad7 protein levels when administered as a gene therapeutic and that it is at least equally effective as the endogenous mouse Smad7 cDNA sequence in mice.

According to yet further aspects, administering a gene therapeutic containing the optimized sequence increased the mass of different muscles in healthy mice whether administered locally or systemically. It also stimulated muscle hypertrophy in mouse models of Duchenne muscular dystrophy and similarly increased muscle strength. These findings indicate that codon-optimization of the human Smad7 cDNA sequence, when incorporated into a vector delivery system, is a novel approach for stimulating muscle mass and function in healthy and dystrophic muscle. Because the amino acid sequence is identical to that produced with the endogenous Smad7 cDNA sequence, these findings further indicate that the codon optimized sequence is equally effective in preventing wasting and excessive ActRIIB signaling with limited risks of off-target effects.

In an aspect, a codon-optimized Smad7 polynucleotide is provided, wherein the codon-optimized polynucleotide is Smad7 having the nucleotide sequence of SEQ ID NO: 1 or a nucleotide sequence having at least 90% identity thereto. The polynucleotide can be of human origin. The codon adaptation index can be increased to the ideal range of 0.8-1.0. One or more of rare tandem repeats, anti-viral motifs, hairpins and negative cis elements can be eliminated. The cDNA stop codon can be changed to TAA. The codons corresponding to residues that are methylated (arginine 57 and arginine 67) can be changed to code for any other amino acid other than lysine, which is optionally methylated. The polynucleotide can be included in a viral vector or a chimeric/hybrid viral vector. That is, a viral vector or a chimeric/hybrid viral vector can comprise a codon-optimized Smad7 polynucleotide, wherein the codon-optimized polynucleotide is Smad7 having the nucleotide sequence of SEQ ID NO: 1 or a nucleotide sequence having at least 90% identity thereto. The chimeric/hybrid viral vector can comprise capsid components selected from one or more of the following adeno-associated virus serotypes: AAV1, AAV2, AAV3, AAV4, AAV5, AAV6, AAV7, AAV8, AAV9, AAV10, AAV11 and/or AAV12. The chimeric/hybrid viral vector can be derived by directed evolution or other artificial selection technique from one or more of the following adeno-associated virus serotypes: AAV1, AAV2, AAV3, AAV4, AAV5, AAV6, AAV7, AAV8, AAV9, AAV10, AAV11 and/or AAV12. The viral vector can be derived from one of the 12 adeno-associated virus serotypes or a chimeric/hybrid viral vector using directed evolution, machine learning or other synthetic biology technique. The codon optimized gene can be flanked by inverted terminal repeat sequences. The viral vector or a chimeric/hybrid viral vector can comprise a muscle-specific promoter, gene regulatory cassette, or enhancer that directs expression of the optimized gene or cDNA in muscle cells. The viral vector or a chimeric/hybrid viral vector can provide expression of the codone-optimized polynucleotide in cardiac muscle cells, skeletal muscle cells, or both. The viral vector or a chimeric/hybrid viral vector can comprise a tissue-specific silencer that limits expression of the Smad7 polynucleotide to muscle cells or to heart cells.

An aspect provides a method of increasing or prolonging Smad7 expression in a subject. The method can comprise using a recombinant viral vector including a codon-optimized Smad7 polynucleotide wherein the codon-optimized polynucleotide is modified to increase the codon adaptation index, remove rare tandem repeats and negative cis elements and/or modify the stop codon relative to the wild-type Smad7 sequence. The codon-optimized Smad7 polynucleotide can be included in a viral vector or a chimeric/hybrid viral vector. The chimeric/hybrid viral vector can comprise capsid components selected from one or more of the following adeno-associated virus serotypes: AAV1, AAV2, AAV3, AAV4, AAV5, AAV6, AAV7, AAV8, AAV9, AAV10, AAV11 and/or AAV12. The viral vector can be derived from one of the 12 adeno-associated virus serotypes or a chimeric/hybrid viral vector using directed evolution, machine learning or other synthetic biology technique. The codon-optimized Smad7 polynucleotide can be flanked by inverted terminal repeat sequences. The codon-optimized Smad7 polynucleotide can be delivered to tissues using a non-viral gene delivery system. The codon-optimized Smad7 polynucleotide can be set forth in SEQ ID NO: 1 or can be a nucleotide sequence having at least 90% identity thereto

An aspect provides a method of enhancing muscle mass and/or strength in a subject, comprising administering to the subject a therapeutically effective amount of a codon-optimized Smad7 polynucleotide, or a viral vector or a chimeric/hybrid viral vector as described herein. The codon-optimized polynucleotide can be Smad7 having the nucleotide sequence of SEQ ID NO: 1 or a nucleotide sequence having at least 90% identity thereto.

An aspect provides a method of enhancing muscle mass and/or strength in a subject, comprising administering to the subject a therapeutically effective amount of the codon-optimized Smad7 polynucleotide, viral vector, or chimeric/hybrid viral vector as described herein to the subject.

An aspect provides a method of enhancing muscle mass and/or strength in a subject for cosmetic reasons, comprising administering to the subject an effective amount of the codon-optimized Smad7 polynucleotide, viral vector, or chimeric/hybrid viral vector as described herein to the subject.

An aspect provides a method of treating muscle wasting in a subject diagnosed with a muscular dystrophy, comprising selecting a subject with a muscular dystrophy and administering to the subject a therapeutically effective amount of the codon-optimized Smad7 polynucleotide, viral vector, or chimeric/hybrid viral vector as described herein.

An aspect provides a method of treating muscle wasting to increase muscle strength and/or muscle volume comprising administering the codon-optimized Smad7 polynucleotide, viral vector, or a chimeric/hybrid viral vector as described herein to a subject.

An aspect provides a method of inhibiting or preventing muscle wasting in a subject, comprising administering to the subject a therapeutically effective amount of the codon-optimized Smad7 polynucleotide, viral vector, or chimeric/hybrid viral vector as described herein to the subject. The muscle wasting can be caused by a chronic disorder. The chronic disorder can comprise a muscular dystrophy, a myopathy, a neurodegenerative disease, cancer, aging (sarcopenia), kidney disease, chronic obstructive pulmonary disorder, chronic infection, AIDS, disuse atrophy, neuromuscular injury, neuropathies, obesity, cardiovascular disease, or a combination of two or more thereof. The muscle wasting can be caused by microgravity stress or prolonged exposure to microgravity and/or space flight. The muscle wasting can comprise wasting of cardiac muscle, skeletal muscle, or both.

In an aspect the codon-optimized Smad7 polynucleotide, viral vector, or chimeric/hybrid viral vector can be delivered via intramuscular or intravenous injections. A single dose or multiple doses can be administered.

### BRIEF DESCRIPTION OF THE DRAWINGS

FIGS. 1A-1C show, according to particular aspects, the characteristics of wild-type and codon-optimized hSmad7 sequences relative to codon position. Both sequences were compared to reference genes expressed in human skeletal muscle. (FIG. 1A) The frequency of conserved codons in wild-type hSmad7 cDNA (black) was considerably less than that in codon-optimized hSmad7 cDNA (gray) across the coding sequence. (FIG. 1B) The percent distribution of codons in computed codon quality groups was higher in the codon-optimized (CO) than wild-type (WT) sequence; higher is better as 100 represents the highest use. (FIG. 1C) The relative GC content by position varied between sequences as large peaks in the wild-type sequence (black line), representing opportunities for 2° structures that can potentially disrupt translation, were removed with optimization (gray line).

FIG. 2 shows, according to particular aspects, an alignment of the wild-type (SEQ ID NO: 4) and codon-optimized hSmad7 cDNA sequences (SEQ ID NO:4). Wild-type hSmad7 cDNA sequence variant 1 (GenBank NM_005904.3) was aligned to the codon-optimized sequence using CLUSTALW (sequences are named on the left, nucleotide positions are numbered on both sides and boxed regions reflect identities). The two sequences were found to be 79% identical.

FIGS. 3A-3F show, according to particular aspects, differences in the predicted mRNA secondary structure of wild-type (WT) and codon-optimized hSmad7 mRNA. Hairpin motifs were separately identified from minimal free energy (MFE) and thermodynamic predictions (FIGS. 3A-3D) using RNA-fold computational software. Secondary structures were then separately modeled from these predictions to produce a single MFE structure for each sequence and a centroid structure generated from multiple sub-optimal predicted foldings using increments above MFE. (FIGS. 3A-3D) Each nucleotide position is illustrated using dot-bracket notation where complementary base pairs are indicated by left and right brackets and unbound nucleotides by dots. Hairpin loops are highlighted in grayscale according to the key and indicate differences between WT and optimized sequences for each model. (FIGS. 3E-3F) Nucleotide positions and base pairings were constructed according to probabilities, which were calculated from positional entropies. Entropy states are represented in grayscale where high energy requirements to eliminate secondary structures are darker. Less compaction and more linearity in the overall sequence is consistent with improved secondary structure.

FIGS. 4A-4D show, according to particular aspects, comparable ability of wild-type and codon-optimized Smad7 cDNA sequences to increase muscle mass. Three recombinant adeno-associated viral vectors with serotype 6 capsids (rAAV6) were generated, each containing either the wild-type mouse or codon-optimized human Smad7 cDNA (mSmad7 or hSmad7). Expression of the mouse sequence was controlled by the ubiquitously active cytomegalovirus (CMV) promoter whereas the human sequences were controlled by either the CMV promoter or the muscle-specific CK8 promoter/regulatory domain, producing the following vectors: rAAV6:CMV-mSmad7 (CMV-m), rAAV6:CMV-hSmad7 (CMV-h) and rAAV6:CK8-hSmad7 (CK8-h). Tibialis anterior (TA) muscles of 2 m.o. mice (n=6/group) were then injected once intramuscularly with 1×10¹⁰ vg of CMV-m, CMV-h or CK8-h. For each mouse, one TA was injected with vector while the contralateral TA was injected with the same volume of saline. (FIG. 4A) After 4 weeks, the absolute mass of treated muscles was greater than that of controls (mean +/−SEM shown). Significant differences between control and treated tissues are indicated by the shown probability levels. (FIG. 4B) The relative difference between limbs was greatest in mice receiving CK8-h (mean +/−SEM shown). Significant differences between vectors are indicated by different letters (p<0.05), same letters denote no difference. (FIG. 4C) Smad7 protein levels were determined by western blotting and were greatest in mice receiving CK8-h. Equal amounts of protein were analyzed for each mouse and GAPDH was used as a loading control. Mean ratios (+/−SEM) of hSmad7:GAPDH are presented as optical density units (ODUs) arbitrarily multiplied by 100. Significant differences between vectors are indicated by different letters (p<0.05), same letters denote no difference. (FIG. 4D) The change in muscle mass is correlated to Smad7 protein levels. A correlation analysis was performed on the Smad7 v. muscle mass relationship using % change data producing the indicated r² and probability values.

FIGS. 5A-5G show, according to particular aspects, comparable ability of wild-type and codon-optimized Smad7 cDNA sequences to stimulate muscle fiber hypertrophy. Tibialis anterior (TA) muscles of 2 m.o. mice (n=6/group) were injected once intramuscularly with 1×10¹⁰ vg of rAAV6:CMV-mSmad7 (CMV-m), rAAV6:CMV-hSmad7 (CMV-h) or rAAV6:CK8-hSmad7 (CK8-h). For each mouse, one TA was injected with vector while the contralateral TA was injected with the same volume of saline. (FIG. 5A) Muscle fiber hypertrophy is readily apparent when comparing treated to control muscle cross-sections from each group. Representative images are shown, nuclei were stained with DAPI (small dots) and sarcolemma with Alexa 647-labeled wheat germ agglutinin (cell outlines). (FIG. 5B) Mean (+/−SEM) muscle fiber size in control and treated muscles, determined by measuring minimum feret diameter, is elevated in all groups, although the difference between control and treated muscles is only significant with CK8-h (statistical differences are indicated by the shown probability levels). (FIGS. 5C-5E) The distribution of fiber sizes was altered with each vector treatment, resulting in fewer small fibers and more large fibers. Fiber sizes were binned into groups (small, <20 mm, medium, 20-70 mm; large, >70 mm; Min, minimal) and significant differences (p<0.05) between control and treated means (−/+SEM) within each size range are represented by asterisks. (FIG. 5F) Relative differences between control and treated muscles in the overall distribution of fiber sizes (minimal feret diameter ranges) for each vector were similar. (FIG. 5G) Binned fiber size relative differences between treated muscles and their respective controls for each vector (small, <20 mm, medium, 20-70 mm; large, >70 mm) were also similar.

FIGS. 6A-6D show, according to particular aspects, that muscle expression of codon-optimized hSmad7 cDNA increases muscle force production. Tibialis anterior (TA) muscles of treated mice were separately injected with saline and rAAV6:CK8-hSmad7 using 2 doses of the latter (1×10⁹ & 1×10¹⁰ vg, 1E9 & 1E10, respectively). Control mice were injected with saline in both TA muscles and dorsiflexor force/torque was assessed in all mice after 8 weeks. (FIG. 6A) Relative differences in mean (−/+SEM) TA mass between treated and contralateral control muscles indicate that rAAV6:CK8-hSmad7 increased TA mass in a dose-dependent manner. (FIGS. 6B-6C) Both doses of rAAV6:CK8-hSmad7 increased absolute dorsiflexion force and to the same relative degree over that of the contralateral control limbs (mean −/+SEM for both). (FIGS. 6A-6C) Different letters between any 2 groups signify statistical significance and asterisks signify differences between contralateral control vs. treated muscles/limbs (p<0.05). (FIG. 6D) Correlation analysis of force-mass relationship for all mice in each dose group indicates that force scales with mass, particularly at the higher dose. Coefficients of determination (r²) and significance (p) of the relationships are inset.

FIGS. 7A-7G show, according to particular aspects, rAAV6:CK8-hSmad7 increases muscle mass and fiber size in a murine model of Duchenne muscular dystrophy (DMD), the mdx mouse. Because DMD pathogenesis develops from birth and has an average age of diagnosis below 4 y.o. in the US, our studies were performed with juvenile mice to mimic the most likely patient development stage for treatment. Wild-type (WT) and mdx neonates were injected R.O. when 1 W.O. with 0, 5×10¹² (5e12), 1.7×10¹³ (1.7e13) and 5×10¹³ (5e13) vg/kg body mass rAAV6:CK8-hSmad7 and terminated after 12 weeks. (FIGS. 7A-7D) The mass of each muscle indicated was enhanced with treatment and in a dose-dependent manner (WT, wild-type; EDL, extensor digitorum longus). Different letters between any 2 groups signify statistical significance (p<0.05) whereas shared letters indicate no difference. (FIG. 7E-7F) Treating with 5×10¹³ vg/kg rAAV6:CK8-hSmad7 shifted the distribution of muscle fiber sizes in the TA muscles of wild-type (WT) and mdx mice to the right due to the presence of more large fibers. This shift was reflected in the mean median fiber sizes among the 19 bins plotted. (FIG. 7G) Comparison of the binned fiber size distribution (small, <30 mm, medium, 30-60 mm; large, >60 mm; Min, minimal) in both WT and mdx mice illustrates a trend of fewer small fibers and more larger fibers with treatment. Significant differences (p<0.05) within each size range are represented by different letters, shared letters signify no difference.

FIGS. 8A-8E show, according to particular aspects, that rAAV6:CK8-hSmad7 does not exacerbate the histophysiology of dystrophic muscle. Wild-type (WT) and mdx neonates were injected R.O. when 1 W.O. with 0, 5×10¹² (5e12), 1.7×10¹³ (1.7e13) and 5×10¹³ (5e13) vg/kg body mass rAAV6:CK8-hSmad7 and terminated after 12 weeks. (FIG. 8A-8B) Serum creatine kinase levels and muscle fibrosis remained elevated in mdx mice regardless of treatment, but were not exacerbated. (FIG. 8C) Muscle central nucleation, a marker of compensatory muscle regeneration due to enhance degeneration, was slightly yet significantly reduced with treatment. (FIGS. 8A-8C) Significant differences are indicated by different letters whereas the same letters indicate no difference. (FIG. 8D) Muscle hypertrophy with rAAV6:CK8-hSmad7 treatment and elevated fibrosis in mdx mice is readily apparent in Masson's tri-chrome-stained sections of TA muscles. Sarcolemmal membranes are dark gray, cytoplasm light gray and collogen thick black. (FIG. 8E) Elevated central nucleation in mdx mice is readily apparent in TA muscle sections stained with wheat germ agglutinin Alex Fluor 488 to outline sarcolemmal membranes and DAPI to label nuclei (light gray dots).

FIGS. 9A-9F show, according to particular aspects, that rAAV6:CK8-hSmad7 enhances muscle function in mdx mice. Wild-type (WT) and mdx neonates were injected R.O. when 1 W.O. with 0, 5×10¹² (5e12), 1.7×10¹³ (1.7e13) or 5×10¹³ (5e13) vg/kg body mass rAAV6:CK8-hSmad7. Plantarflexion force/torque was quantified after 12 weeks using an in vivo whole-animal murine system with motor, arm and force transducer from Aurora Scientific. Assays were performed on anesthetized mice (n=6/group) with increasing stimulation frequencies as indicated. Dashed lines in FIGS. 9A, 8C and 8E frame the physiologically relevant range of stimulation frequencies. (FIG. 9A) Total force generating capacity of mdx plantarflexor muscles was elevated with treatment and in a dose-dependent manner. Significant differences between mdx controls (0) and mdx 1e14 are indicated by the probability levels shown. (FIG. 9B) Force at 80 Hz, which produced maximal to near-maximal responses for all contractile metrics, mirrored the dose-responsive increases across the frequency range tested. Significant differences are indicated by different letters, the same letters indicate no difference. (FIGS. 9C-9F) The contraction rate was similarly enhanced by rAAV6:CK8-hSmad7 under the conditions described above and although the relaxation rate was enhanced, the dose-dependency was less evident. Significant differences are again indicated by probability levels or different letters as before.

FIGS. 10A-10D show, according to particular aspects, that rAAV6:CK8-hSmad7 stimulates changes in muscle force that scale to those in muscle mass. (FIGS. 10A-10C) Total force, contraction rate and relaxation rate of all mdx mice were correlated to their corresponding plantarflexor muscle mass. Both force and contraction rate were positively and significantly correlated to plantar mass whereas relaxation rate was not. Pearson correlation coefficients (r) and two-tailed probability levels (p) for the relationship are inset in each graph. (FIG. 10D) Differences in total force at 80 Hz were lost when normalized to plantar mass. Significant differences are indicated by different letters, the same letters indicate no difference.

FIG. 11 shows, according to particular aspects, the map of the plasmid (pAAV-MCS:CK8-hSmad7.opt, see also SEQ ID NO: 2) used to create AAV6:CK8-hSmad7. Total plasmid size is 5824 bp (inset numbering). Inner arrows represent open reading frames for codon-optimized human smad7 cDNA (hSMAD7) and ampicillin resistance (AmpR) cDNA. Outer arrows indicate transcription start sites for each. Non-coding elements include the CK8 regulatory cassette, which is derived from the muscle creatine kinase promoter and is functionally active only in striated muscle (Himeda et al., Methods Mol Biol 709, 3-19, 2011; Bengtsson et al., Nat Commun 8, 14454, 2017). Other non-coding elements include an intron and the human growth hormone (hGH) poly-adenylation sequence. Flanking this expression construct are two AAV2 inverted terminal repeat (ITR) sequences.

FIG. 12 shows, according to particular aspects, the pAAV-MCS:CK8-hSmad7.opt sequence that corresponds to the plasmid map in FIG. 11 (SEQ ID NO: 2). Nucleotides are blocked in 10 bp segments with 100 bp/line (numbers on right). Subsequences corresponding to the elements are mapped in clock-wise order: left ITR (underlined, 1-141 bp), CK8e promoter (italics, 163-612 bp; SEQ ID NO:5 AGCTAGACTA GCATGCTGCC CATGTAAGGA GGCAAGGCCT GGGGACACCC GAGATGCCTG GTTATAATTA ACCCAGACAT GTGGCTGCCC CCCCCCCCCC AACACCTGCT GCCTCTAAAA ATAACCCTGC ATGCCATGTT CCCGGCGAAG GGCCAGCTGT CCCCCGCCAG CTAGACTCAG CACTTAGTTT AGGAACCAGT GAGCAAGTCA GCCCTTGGGG CAGCCCATAC AAGGCCATGG GGCTGGGCAA GCTGCACGCC TGGGTCCGGG GTGGGCACGG TGCCCGGGCA ACGAGCTGAA AGCTCATCTG CTCTCAGGGG CCCCTCCCTG GGGACAGCCC CTCCTGGCTA GTCACACCCT GTAGGCTCCT CTATATAACC CAGGGGCACA GGGGCTGCCC TCATTCTACC ACCACCTCCA CAGCACAGAC AGACACTCAG GAGCCAGCCA GC), β-globin intron (italics, 626-1118 bp; SEQ ID NO:6 GGATTCGAAT CCCGGCCGGG AACGGTGCAT TGGAACGCGG ATTCCCCGTG CCAAGAGTGA CGTAAGTACC GCCTATAGAG TCTATAGGCC CACAAAAAAT GCTTTCTTCT TTTAATATAC TTTTTTGTTT ATCTTATTTC TAATACTTTC CCTAATCTCT TTCTTTCAGG GCAATAATGA TACAATGTAT CATGCCTCTT TGCACCATTC TAAAGAATAA CAGTGATAAT TTCTGGGTTA AGGCAATAGC AATATTTCTG CATATAAATA TTTCTGCATA TAAATTGTAA CTGATGTAAG AGGTTTCATA TTGCTAATAG CAGCTACAAT CCAGCTACCA TTCTGCTTTT ATTTTATGGT TGGGATAAGG CTGGATTATT CTGAGTCCAA GCTAGGCCCT TTTGCTAATC ATGTTCATAC CTCTTATCTT CCTCCCACAG CTCCTGGGCA ACGTGCTGGT CTGTGTGCTG GCCCATCACT TTGGCAAAGA ATTGGG), codon-optimized hSmad7 ORF (bold, 1199-2479 bp) (SEQ ID NO:1), hGH poly-A (italics, 2569-3048 bp), right ITR (underlined, 3087-3228 bp) and ampR (light gray, 4144-5004 bp).

### SEQUENCE LISTING

The nucleic and amino acid sequences included in the sequence listing below are shown using standard letter abbreviations for nucleotide bases and amino acids as defined in 37 C.F.R. 1.822. Only one strand of each nucleic acid sequence is shown, but the complementary strand is understood as included by any reference to the displayed strand.
SEQ ID NO: 1 is the nucleic acid sequence of the codon-optimized human Smad7 cDNA.
SEQ ID NO: 2 is the nucleic acid sequence of the codon-optimized human Smad7 AAV vector pAAV-MCS:CK8-hSmad7.opt
SEQ ID NO: 3 is the amino acid sequence of human Smad7 protein.
SEQ ID NO: 4 is the nucleic acid sequence of the wild-type human Smad7 cDNA.
SEQ ID NO: 5 is the nucleic acid sequence of the CK8e promoter.
SEQ ID NO: 6 is the nucleic acid sequence of the MHCK7 promoter.
SEQ ID NO: 7 is the nucleic acid sequence of the CMV promoter
SEQ ID NO: 8 is GGAGGAGGAGGA (direct repeat)

### I. Abbreviations

AAV adeno-associated virus
ActRII Type II activin receptor
ALK activin like kinase
CAI codon adaptation index
CFD codon frequency distribution
CK8 creatine kinase 8 promoter
CMV cytomegalovirus
DMD Duchenne muscular dystrophy
EDL extensor digitalis longus
GC guanine and cytosine
IEE integration efficiency element
IM intramuscular
ITR inverted terminal repeat
IV intravenous
kg kilograms
mo month(s) old
ORF open reading frame
rAAV recombinant AAV
ro retro-orbital
SEM standard error of the mean
TA tibialis anterior
TRS terminal resolution site
vg vector genomes
wo week(s) old
WT wild-type

### II. Terms and Methods

Unless otherwise noted, technical terms are used according to conventional usage. Definitions of common terms in molecular biology may be found in Benjamin Lewin, Genes V, published by Oxford University Press, 1994 (ISBN 0-19-854287-9); Kendrew et al. (eds.), The Encyclopedia of Molecular Biology, published by Blackwell Science Ltd., 1994 (ISBN 0-632-02182-9); and Robert A. Meyers (ed.), Molecular Biology and Biotechnology: A Comprehensive Desk Reference, published by VCH Publishers, Inc., 1995 (ISBN 1-56081-569-8).

In order to facilitate review of the various embodiments of the disclosure, the following explanations of specific terms are provided:

Adeno-associated virus (AAV): A small, replication-defective, non-enveloped virus that infects mammalian species. Eleven naturally occurring AAV serotypes have been identified to date (AAV1-AAV11) while several additional derivatives of these vectors have been generated artificially, each with unique tropism for specific tissue types. AAV is not known to cause disease and elicits a very mild immune response. Gene therapy vectors that utilize AAV can infect dividing and quiescent cells as well as undifferentiated (immature) and differentiated (mature) cells depending on the specific AAV serotype in use. AAV genomes can persist in an extrachromosomal state without integrating into the genome of the host cell. These features make AAV an attractive viral vector for gene therapy.

Administration/Administer: To provide or give a subject an agent, such as a therapeutic agent (e.g., a recombinant AAV), by any effective route. Exemplary routes of administration include, but are not limited to, injection (such as subcutaneous, intramuscular, intradermal, intraperitoneal, intravenous and retro-orbital), oral, intraductal, sublingual, rectal, transdermal, intranasal, vaginal and inhalation routes.

Codon-optimized: A “codon-optimized” nucleic acid refers to a nucleic acid sequence that has been altered such that the codons are optimal for expression in a particular system (e.g., particular species, group of species or tissue). For example, a nucleic acid sequence can be optimized for expression in mammalian cells or in a particular tissue of a specific mammalian species (e.g., human muscle cells). Codon optimization does not alter the amino acid sequence of the encoded protein.

Enhancer: A nucleic acid sequence that increases the rate of transcription by increasing the activity of a promoter.

Infective: A virus or vector is “infective” when it transduces a cell, replicates, and (without the benefit of any complementary virus or vector) spreads progeny vectors or viruses to other cells in an organism or cell culture, where the progeny vectors or viruses have the same ability to reproduce and spread throughout the organism or cell culture. For example, a nucleic acid encoding an adeno-associated viral particle is not infective if the nucleic acid cannot be packaged (e.g., if the adeno-associated viral particle lacks a packaging site), even though the nucleic acid can be used to transduce a cell. Similarly, an adeno-associated viral nucleic acid packaged by an adeno-associated viral particle is not infective if it does not encode the adeno-associated viral capsid proteins used for packaging.

Intron: A stretch of DNA within a gene that does not contain coding information for a protein. Introns are removed before translation of a messenger RNA.

Inverted terminal repeat (ITR): Symmetrical nucleic acid sequences in the genome of adeno-associated viruses required for efficient replication. ITR sequences are located at each end of the AAV DNA genome. The ITRs serve as the origins of replication for viral DNA synthesis and are essential cis components for generating AAV integrating vectors.

Isolated: An “isolated” biological component (such as a nucleic acid molecule, protein, virus or cell) has been substantially separated or purified away from other biological components in the cell or tissue of the organism, or the organism itself, in which the component naturally occurs, such as other chromosomal and extra-chromosomal DNA and RNA, proteins and cells. Nucleic acid molecules and proteins that have been “isolated” include those purified by standard purification methods. The term also embraces nucleic acid molecules and proteins prepared by recombinant expression in a host cell as well as chemically synthesized nucleic acid molecules and proteins.

Mammal: This term includes both human and non-human mammals. Similarly, the term “subject” includes both human and veterinary subjects.

Muscle/striated muscle: Although there are three types of muscle, the term “muscle” refers to skeletal muscle only and not cardiac or smooth muscle unless otherwise noted. Striated muscle includes skeletal and cardiac, but not smooth.

Operably linked: A first nucleic acid sequence is operably linked with a second nucleic acid sequence when the first nucleic acid sequence is placed in a functional relationship with the second nucleic acid sequence. For instance, a promoter is operably linked to a coding sequence if the promoter affects the transcription or expression of the coding sequence. Generally, operably linked DNA sequences are contiguous and, where necessary to join two protein-coding regions, in the same reading frame.

Packaging cell: A cell that provides packaging functions in trans for a gene introduced into a cell with a transfer vector, but which does not encapsidate its own genome.

Packaging Vector: Packaging vector nucleic acids lack the nucleic acids necessary for packaging of a DNA corresponding to the packaging vector nucleic acid into an adeno-associated viral capsid. That is, packaging vector nucleic acids are not themselves encapsidated in the viral particles that they encode (i.e., they are not infective). The packaging vector optionally includes all of the components necessary for production of viral particles, or optionally includes a subset of the components necessary for viral packaging. For instance, a packaging cell may be transformed with more than one packaging vector, each of which has a complementary role in the production of an adeno-associated viral particle.

Two (or more) viral-based packaging vectors are “complementary” when they together encode all of the functions necessary for adeno-associated virus packaging, and when each individually does not encode all of the functions necessary for packaging. For example, when two vectors transduce a single cell and together encode the information for production of adeno-associated virus packaging particles, the two vectors are “complementary.” The use of complementary vectors increases the safety of any packaging cell made by transformation with a packaging vector by reducing the possibility that a recombination event will produce an infective virus.

Pharmaceutically acceptable carrier: The pharmaceutically acceptable carriers (vehicles) useful in this disclosure are conventional. Remington's Pharmaceutical Sciences, by E. W. Martin, Mack Publishing Co., Easton, PA, 15th Edition (1975), describes compositions and formulations suitable for pharmaceutical delivery of one or more therapeutic compounds, molecules or agents.

In general, the nature of the carrier will depend on the particular mode of administration being employed. For instance, parenteral formulations usually comprise injectable fluids that include pharmaceutically and physiologically acceptable fluids such as water, physiological saline, balanced salt solutions, aqueous dextrose, glycerol or the like as a vehicle. For solid compositions (for example, powder, pill, tablet, or capsule forms), conventional non-toxic solid carriers can include, for example, pharmaceutical grades of mannitol, lactose, starch, or magnesium stearate. In addition to biologically-neutral carriers, pharmaceutical compositions to be administered can contain minor amounts of non-toxic auxiliary substances, such as wetting or emulsifying agents, preservatives, and pH buffering agents and the like, for example sodium acetate or sorbitan monolaurate.

Preventing, treating or ameliorating a disease: “Preventing” a disease (such as muscular dystrophy) or a symptom associated with a disease (such as muscle wasting) refers to inhibiting the full development of a disease or symptom. “Treating” refers to a therapeutic intervention that ameliorates a sign or symptom of a disease or pathological condition after it has begun to develop. “Ameliorating” refers to the reduction in the number or severity of signs or symptoms of a disease.

Promoter: A region of DNA that directs/initiates transcription of a nucleic acid (e.g., a gene). A promoter includes necessary nucleic acid sequences near the start site of transcription. Typically, promoters are located near the genes they transcribe. A promoter also optionally includes distal enhancer or repressor elements that can be located near (e.g., dozens of base pairs) or far (e.g., several thousand base pairs) from the transcription start site.

Purified: The term “purified” does not require absolute purity; rather, it is intended as a relative term. For example, a purified peptide, protein, virus, or other active compound is one that is isolated in whole or in part from naturally associated proteins and other contaminants. In certain embodiments, the term “substantially purified” refers to a peptide, protein, virus or other active compound that has been isolated from a cell, cell culture medium, or other crude preparation and subjected to fractionation to remove various components of the initial preparation, such as proteins, cellular debris, and other components.

Recombinant: A recombinant nucleic acid molecule is one that has a sequence that is not naturally occurring or has a sequence that is made by an artificial combination of two otherwise separated segments of sequence. This artificial combination can be accomplished by chemical synthesis or by the artificial manipulation of isolated segments of nucleic acid molecules, such as by genetic engineering techniques.

Similarly, a recombinant virus is a virus comprising sequence (such as genomic sequence) that is non-naturally occurring or made by artificial combination of at least two sequences of different origin. The term “recombinant” also includes nucleic acids, proteins and viruses that have been altered solely by addition, substitution, or deletion of a portion of a natural nucleic acid molecule, protein or virus. As used herein, “recombinant AAV” refers to an AAV particle in which a recombinant nucleic acid molecule (such as a recombinant nucleic acid molecule encoding codon-optimized human Smad7 cDNA) has been packaged.

Sequence Identity: The identity or similarity between two or more nucleic acid sequences, or two or more amino acid sequences, is expressed in terms of the identity or similarity between the sequences. Sequence identity can be measured in terms of percentage identity; the higher the percentage, the more identical the sequences are. Sequence similarity can be measured in terms of percentage similarity (which takes into account conservative amino acid substitutions); the higher the percentage, the more similar the sequences are. Homologs of nucleic acid or amino acid sequences possess a relatively high degree of sequence identity/similarity when aligned using standard methods. This homology is more significant when the orthologous proteins or cDNAs are derived from species that are more closely related (such as human and mouse sequences), compared to species more distantly related (such as human and C. elegans sequences).

Methods for aligning and comparing sequences are well known in the art as various programs and alignment algorithms have been developed. Notable is Altschul et al., J. Mol. Biol. 215:403-10, 1990, which presents a detailed consideration of sequence alignment methods and homology calculations as well as a description of the Basic Local Alignment Search Tool (BLAST). This particular tool is available at several sources including the National Center for Biological Information (NCBI) and on the internet. It is also used in connection with the sequence analysis programs blastp, blastn, blastx, tblastn and tblastx. Additional information can be found at the NCBI web site.

Serotype: A group of closely related microorganisms (such as viruses) distinguished by a characteristic set of antigens.

Subject: Living multi-cellular vertebrate organisms, a category that includes human and non-human mammals.

Synthetic: Produced by artificial means in a laboratory, for example a synthetic nucleic acid can be chemically synthesized in a laboratory.

Therapeutically effective amount: A quantity of a specified pharmaceutical or therapeutic agent (e.g., a recombinant AAV) sufficient to achieve a desired effect in a subject, or in a cell, being treated with the agent. The effective amount of the agent will be dependent on several factors, including, but not limited to the subject or cells being treated, and the manner of administration of the therapeutic composition.

Treating or treatment: Includes the application or administration of a composition to a subject, or application or administration of a composition to a cell or tissue from a subject has symptoms of muscle wasting, as with muscular dystrophy, with the purpose of curing, healing, alleviating, relieving, altering, remedying, ameliorating, improving, or affecting the disease or condition, the symptom of the disease or condition, or the risk of the disease or condition.

Vector: A vector is a nucleic acid molecule allowing insertion of foreign nucleic acid without disrupting the ability of the vector to replicate and/or integrate in a host cell. A vector can include nucleic acid sequences that permit it to replicate in a host cell, such as an origin of replication. A vector can also include one or more selectable marker genes and other genetic elements. An expression vector is a vector that contains the necessary regulatory sequences to allow transcription and translation of inserted gene or genes. In some embodiments herein, the vector is an AAV vector.

### DETAILED DESCRIPTION OF EXEMPLARY ASPECTS

Isolated skeletal muscle atrophy or systemic muscle wasting, identical conditions that differ only by scale, significantly contribute to the pathogenesis of many disease states and conditions (Cohen et al., Nat Rev Drug Discov 14, 58-74, 2015; Bodine and Baehr Am J Physiol Endocrinol Metab 307, E469-484, 2014). Cardiac muscle wasting also occurs in some of these conditions as the mechanisms of wasting are conserved in both muscle types (i.e., striated muscle). Wild-type Smad7 cDNA includes secondary structure-causing motifs capable of limiting Smad7 protein translation. The strategy described herein is an improved design that not only lacks these motifs, but enhances the overall translational potential of the Smad7 cDNA. We therefore contend that this strategy could translate to interventions capable of reducing the disability, morbidity and mortality associated with muscular dystrophies and myopathies as well as with other muscle wasting disease states, especially as several gene-based therapies are currently being developed for neuromuscular disorders and non-muscle-diseases (Mendell et al., Mol Ther 29, 464-488, 2021; Fortunato et al., Neuromuscul Disord 31, 1013-1020, 2021).

According to particular aspects and given that elevated circulating concentrations or local production of myostatin, activin and other ActRII ligands can cause muscle wasting and have been associated with chronic conditions where muscle wasting occurs (Rodgers and Ward Endocr Rev 2021), considerable effort has been invested in developing interventions that can mitigate the harmful effects of excessive ActRII signaling. Most of these interventions target the responsible ligands in the circulation or the extracellular environment, although their feasibility remains in question due to potential off-target effects associated with inhibition at the extracellular level (Rodgers and Ward Endocr Rev 2021; Campbell et al., Muscle Nerve 55, 458-464, 2017; Garito et al., Clin Endocrinol (Oxf) 88, 908-919, 2018).

To circumvent these problems and to improve upon use of wild-type Smad7 cDNA, we investigated whether inhibiting ActRII action in muscle by overexpressing codon-optimized hSmad7 represents a strategy to inhibit muscle wasting and build muscle mass without binding ActRII ligands. This approach increases intracellular hSmad7 levels and thereby attenuates ActRII signaling from inside the cell rather than preventing extracellular ligands from binding ActRII outside the cell. We found that healthy muscles treated with rAAV6:CK8-hSmad7 were larger and stronger than untreated muscles. This was also true for dystrophic muscles treated with rAAV6:CK8-hSmad7. The gene therapeutic was effective with different routes of administration, at different doses and in young and adult mice. These results demonstrate the utility of our approach for dissociating the activity of key intracellular signaling processes involved in maintaining muscle mass from the effects of ligands that utilize these pathways to promote muscle wasting. Moreover, the efficacy of rAAV6:CK8e-hSmad7 is superior to the use of wild-type Smad7 yet both approaches are more specific to striated muscle than any attenuator of ActRII ligands and for this reason, they are much less likely to produce off-target effects.

According to particular aspects, IV administration of an rAAV6 gene therapeutic that overexpresses Smad7 in mice also prevents the wasting of cardiac muscle. The overexpressing of codon-optimized hSmad7 would have identical actions as the amino acid sequences of wild-type and codon-optimized hSmad7 proteins are identical. Note that expression of myostatin and activin A is associated with heart disease and that physiological hypertrophy and enhanced cardiac contractility and Ca2+ handling all occur in myostatin-null hearts and with ActRII attenuation Thus, systemic administration of viral vectors capable of overexpressing codon-optimized hSmad7 in the myocardium have the potential to repress ActRIIB-mediated atrophic signaling in the heart.

In an aspect, the codons corresponding to residues that are methylated (i.e., arginine 57, or arginine 67, or both arginine 57 and arginine 67) in Smad7 are changed to code for any other amino acid other than lysine (e.g., Ala, Art, Asn, Asp, Cys, Glu, Gln, Gly, His, Ile, Leu, Met, Phe, Pro, Ser, Thr, Trp, Tyr, or Val). The substituted amino acids at position 57 or 67 or both 57 and 67 can optionally be methylated.

AAVs belong to the family Parvoviridae and the genus Dependovirus. They are small, non-enveloped viruses that packages a linear, single-stranded DNA genome. Both sense and antisense strands of AAV DNA are packaged into AAV capsids with equal frequency.

The AAV genome is characterized by two inverted terminal repeats (ITRs) that flank two open reading frames (ORFs). In the AAV2 genome, for example, the first 125 nucleotides of the ITR are a palindrome, which folds upon itself to maximize base pairing and forms a T-shaped hairpin structure. The other 20 bases of the ITR, called the D sequence, remain unpaired. The ITRs are cis-acting sequences important for AAV DNA replication; the ITR is the origin of replication and serves as a primer for second-strand synthesis by DNA polymerase. The double-stranded DNA formed during this synthesis, which is called replicating-form monomer, is used for a second round of self-priming replication and forms a replicating-form dimer. These double-stranded intermediates are processed via a strand displacement mechanism, resulting in single-stranded DNA used for packaging and double-stranded DNA used for transcription. Located within the ITR are the Rep binding elements and a terminal resolution site (TRS). These features are used by the viral regulatory protein Rep during AAV replication to process the double-stranded intermediates. In addition to their role in AAV replication, the ITR is also essential for AAV genome packaging, transcription, negative regulation under non-permissive conditions, and site-specific integration (Daya and Berns, Clin Microbiol Rev 21(4):583-593, 2008).

The left ORF of AAV contains the Rep gene, which encodes four proteins—Rep 78, Rep 68, Rep 52 and Rep 40. The right ORF contains the Cap gene, which produces three viral capsid proteins (VP1, VP2 and VP3). The AAV capsid contains 60 viral capsid proteins arranged into an icosahedral symmetry. VP1, VP2 and VP3 are present in a 1:1:10 molar ratio (Daya and Berns, Clin Microbiol Rev 21(4):583-593, 2008).

AAV is currently one of the most frequently used viruses for gene therapy. Although AAV infects humans and some other primate species, it is not known to cause disease and elicits a very mild immune response. Gene therapy vectors that utilize AAV can infect both dividing and quiescent cells and persist in an extrachromosomal state without integrating into the genome of the host cell. Because of the advantageous features of AAV, the present disclosure contemplates the use of AAV for the recombinant nucleic acid molecules and methods disclosed herein.

AAV possesses several desirable features for a gene therapy vector, including the ability to bind and enter target cells, enter the nucleus, the ability to be expressed in the nucleus for a prolonged period of time, and low toxicity. However, the small size of the AAV genome limits the size of heterologous DNA that can be incorporated. To minimize this problem, AAV vectors have been constructed that do not encode Rep and the integration efficiency element (IEE). The ITRs are retained as they are cis signals required for packaging (Daya and Berns, Clin Microbiol Rev 21(4):583-593, 2008).

Methods for producing rAAV suitable for gene therapy are well known in the art (see, for example, U.S. Patent Application Nos. 2012/0100606; 2012/0135515; 2011/0229971; and 2013/0072548; and Ghosh et al., Gene Ther 13(4):321-329, 2006), and can be utilized with the recombinant nucleic acid molecules, vectors and methods disclosed herein.

### Isolated Polynucleotides Comprising Transgene Sequences

The present disclosure provides isolated polynucleotides comprising at least one transgene nucleic acid molecule. As used herein, the terms “nucleic acid sequence” and “polynucleotide” are used interchangeably to refer to a polymeric form of nucleotides of any length, either ribonucleotides or deoxyribonucleotides. Thus, this term includes, but is not limited to, single-, double-, or multi-stranded DNA or RNA, genomic DNA, cDNA, DNA-RNA hybrids, or a polymer comprising, consisting essentially of, or consisting of purine and pyrimidine bases or other natural, chemically or biochemically modified, non-natural, or derivatized nucleotide bases.

A “gene” refers to a polynucleotide containing at least one open reading frame (ORF) that is capable of encoding a particular polypeptide or protein. A “gene product” or, alternatively, a “gene expression product” refers to the amino acid sequence (e.g., peptide or polypeptide) generated when a gene is transcribed and translated.

In some aspects, a transgene nucleic acid molecule can comprise a nucleic acid sequence encoding a Smad7 polypeptide, or at least one fragment thereof. In some aspects, a transgene nucleic acid molecule can comprise a nucleic acid sequence encoding a biological equivalent of an Smad7 polypeptide.

In some aspects, a Smad7 polypeptide comprises, consists essentially of, or consists of an amino acid sequence at least 65%, 70%, 75%, 80%, 85%, 90%, 95%, 96%, 97%, 98%, 99% or 100% (or any percentage in between) identical to the amino acid sequence set forth in SEQ ID NO: 3, or a fragment thereof. In some aspects, a Smad7 polypeptide comprises, consists essentially of, or consists of an amino acid sequence at least 65%, 70%, 75%, 80%, 85%, 90%, 95%, 96%, 97%, 98%, 99% or 100% (or any percentage in between) identical to at least one portion of the amino acid sequence set forth in SEQ ID NO: 3, or a fragment thereof. In some embodiments, the fragment is a functional fragment, e.g., a fragment that retains at least one function of wildtype Smad7.

In some aspects, a nucleic acid sequence encoding a Smad7 polypeptide comprises, consists essentially of, or consists of a nucleic acid sequence at least 65%, 70%, 75%, 80%, 85%, 90%, 95%, 96%, 97%, 98%, 99% or 100% (or any percentage in between) identical to the nucleic acid sequence set forth in SEQ ID NO:1.

In some aspects, the nucleic acid sequence encoding a Smad7 polypeptide can be a codon optimized nucleic acid sequence that encodes for a Smad7 polypeptide. A codon optimized nucleic acid sequence encoding a Smad7 polypeptide can comprise, consist essentially of, or consist of a nucleic acid sequence that is no more than 65%, 70%, 75%, 80%, 85%, 90%, 95%, 96%, 97%, 98%, 99% (or any percentage in between) identical to the wildtype human nucleic acid sequence encoding a Smad7 poly-peptide. A wildtype human nucleic acid sequence encoding a Smad7 polypeptide is a nucleic acid sequence that encodes a Smad7 polypeptide in a human genome. Exemplary wildtype human nucleic acid sequence encoding a Smad7 peptide is set forth in SEQ ID NO:4. An exemplary wildtype Smad7 polypeptide is set forth in SEQ ID NO:3. An exemplary codon optimized sequence encoding Smad7 is set forth in SEQ ID NO:1.

In some aspects, a codon optimized nucleic acid sequence encoding a Smad7 polypeptide, such as SEQ ID NO:1, can comprise no donor splice sites. In some aspects, a codon optimized nucleic acid sequence encoding a Smad7 polypeptide can comprise no more than about one, or about two, or about three, or about four, or about five, or about six, or about seven, or about eight, or about nine, or about ten donor splice sites. In some aspects, a codon optimized nucleic acid sequence encoding a Smad7 polypeptide comprises at least one, or at least two, or at least three, or at least four, or at least five, or at least six, or at least seven, or at least eight, or at least nine, or at least ten fewer donor splice sites as compared to the wildtype human nucleic acid sequence encoding a Smad7 polypeptide. Without wishing to be bound by theory, the removal of donor splice sites in the codon optimized nucleic acid sequence can unexpectedly and unpredictably increase expression of a Smad7 polypeptide in vivo, as cryptic splicing is prevented. Moreover, cryptic splicing may vary between different subjects, meaning that the expression level of a Smad7 polypeptide comprising donor splice sites can unpredictably vary between different subjects.

In some aspects, a codon optimized nucleic acid sequence encoding a Smad7 polypeptide, such as SEQ ID NO:1, can have a GC content that differs from the GC content of a wildtype human nucleic acid sequence encoding a Smad7 polypeptide. In some aspects, the GC content of a codon optimized nucleic acid sequence encoding a Smad7 polypeptide is more evenly distributed across the entire nucleic acid sequence, as compared to a wildtype human nucleic acid sequence encoding a Smad7 polypeptide. Without wishing to be bound by theory, by more evenly distributing the GC content across the entire nucleic acid sequence, the codon optimized nucleic acid sequence exhibits a more uniform melting temperature (“Tm”) across the length of the transcript. The uniformity of melting temperature results unexpectedly in increased expression of the codon optimized nucleic acid in a human subject, as transcription and/or translation of the nucleic acid sequence occurs with less stalling of the polymerase and/or ribosome.

In some aspects, a codon optimized nucleic acid sequence encoding a Smad7 polypeptide, such as SEQ ID NO:1, exhibits at least 5%, at least 10%, at least 20%, at least 30%, at least 50%, at least 75%, at least 100%, at least 200%, at least 300%, at least 500%, or at least 1000% increased expression in a human subject relative to a wild-type or non-codon optimized nucleic acid sequence encoding a Smad7 polypeptide.

In some aspects, a Smad7 polypeptide can further comprise a protein tag. Without wishing to be bound by theory, the inclusion of a protein tag can allow for the detection and/or visualization of an exogenous Smad7 polypeptide. Examples of protein tags include Myc tags, poly-histidine tags, FLAG-tags, HA-tags, SBP-tags or any other protein tag known in the art.

### Delivery

A polynucleotide disclosed herein can be delivered to a cell or tissue using a gene delivery vehicle. “Gene delivery,” “gene transfer,” “transducing,” and the like as used herein, are terms referring to the introduction of an exogenous polynucleotide (sometimes referred to as a “transgene”) into a host cell, irrespective of the method used for the introduction. Such methods include a variety of well-known techniques such as vector-mediated gene transfer (by, e.g., viral infection/transfection, or various other protein-based or lipid-based gene delivery complexes) as well as techniques facilitating the delivery of “naked” polynucleotides (such as electroporation, “gene gun” delivery and various other techniques used for the introduction of polynucleotides). The introduced polynucleotide may be stably or transiently maintained in the host cell. Stable maintenance typically requires that the introduced polynucleotide either contains an origin of replication compatible with the host cell or integrates into a replicon of the host cell such as an extrachromosomal replicon (e.g., a plasmid) or a nuclear or mitochondrial chromosome. A number of vectors are known to be capable of mediating transfer of genes to mammalian cells, as is known in the art and described herein.

### AAV Vectors

In some aspects, isolated polynucleotides comprising at least one transgene nucleic acid molecule described herein can be a recombinant AAV (rAAV) vector.

A vector is a nucleic acid comprising, consisting essentially of, or consisting of an intact replicon such that the vector can be replicated when placed within a cell, for example by a process of transfection, infection, or transformation. Once inside a cell, a vector can replicate as an extrachromosomal (episomal) element or can be integrated into a host cell chromosome. Vectors can include nucleic acids derived from retroviruses, adenoviruses, herpesvirus, baculoviruses, modified baculoviruses, papovaviruses, or otherwise modified naturally occurring viruses. Exemplary non-viral vectors for delivering nucleic acid include naked DNA; DNA complexed with cationic lipids, alone or in combination with cationic polymers; anionic and cationic liposomes; DNA-protein complexes and particles comprising, consisting essentially of, or consisting of DNA condensed with cationic polymers such as heterogeneous polylysine, defined-length oligopeptides, and polyethyleneimine, in some cases contained in liposomes; and the use of ternary complexes comprising, consisting essentially of, or consisting of a virus and polylysine-DNA.

Vectors can contain both a promoter and a cloning site into which a polynucleotide can be operatively linked. Such vectors are capable of transcribing RNA in vitro or in vivo and are commercially available from sources such as Agilent Technologies (Santa Clara, Calif) and Promega Biotech (Madison, Wis.). To optimize expression and/or in vitro transcription, it may be necessary to remove, add or alter 5′ and/or 3′ untranslated portions of cloned transgenes to eliminate extra, potential inappropriate alternative translation initiation codons or other sequences that may interfere with or reduce expression, either at the level of transcription or translation. Alternatively, consensus ribosome binding sites can be inserted immediately 5′ of the start codon to enhance expression.

An rAAV vector can comprising, consisting essentially of, or consisting of one or more transgene nucleic acid molecules and one or more AAV inverted terminal repeat sequences (ITRs). Such AAV vectors can be replicated and packaged into infectious viral particles when present in a host cell that provides the functionality of rep and cap gene products; for example, by transfection of the host cell. In some aspects, AAV vectors contain a promoter, at least one nucleic acid that can encode at least one protein or RNA, and/or an enhancer and/or a terminator within the flanking ITRs that is packaged into the infectious AAV particle. The encapsidated nucleic acid portion can be referred to as the AAV vector genome. Plasmids containing rAAV vectors can also contain elements for manufacturing purposes, e.g., antibiotic resistance genes, origin of replication sequences etc., but these are not encapsidated and thus do not form part of the AAV particle.

In some aspects, an rAAV vector can comprise at least one transgene nucleic acid molecule. In some aspects, an rAAV vector can comprise at least one AAV inverted terminal (ITR) sequence. In some aspects, an rAAV vector can comprise at least one promoter sequence. In some aspects, an rAAV vector can comprise at least one enhancer sequence. In some aspects, an rAAV vector can comprise at least one polyA sequence. In some aspects, an rAAV vector can comprise a RepCap sequence.

In some aspects, an rAAV vector can comprise a first AAV ITR sequence, a promoter sequence, a transgene nucleic acid molecule and a second AAV ITR sequence. In some aspects, an rAAV vector can comprise, in the 5′ to 3′ direction, a first AAV ITR sequence, a promoter sequence, a transgene nucleic acid molecule and a second AAV ITR sequence.

In some aspects, an rAAV vector can comprise a first AAV ITR sequence, a promoter sequence, a transgene nucleic acid molecule, a polyA sequence and a second AAV ITR sequence. In some aspects, an rAAV vector can comprise, in the 5′ to 3′ direction, a first AAV ITR sequence, a promoter sequence, a transgene nucleic acid molecule, a polyA sequence and a second AAV ITR sequence.

In some aspects, an rAAV vector can comprise more than one transgene nucleic acid molecule. In some aspects, an rAAV vector can comprise at least two transgene nucleic acid molecules, such that the rAAV vector comprises a first transgene nucleic acid molecule and an at least second transgene nucleic acid molecule. In some aspects, the first and the at least second transgene nucleic acid molecule can comprise the same nucleic acid sequence. In some aspects, the first and the at least second transgene nucleic acid molecules can comprise different nucleic acid sequences. In some aspects, the first and the at least second transgene nucleic acid sequences can be adjacent to each other.

In some aspects, an rAAV vector can comprise more than one promoter sequence. In some aspects, an rAAV vector can comprise at least two promoter sequences, such that the rAAV vector comprises a first promoter sequence and an at least second promoter sequence. In some aspects, the first and the at least second promoter sequences can comprise the same sequence. In some aspects, the first and the at least second promoter sequences can comprise different sequences. In some aspects, the first and the at least second promoter sequences can be adjacent to each other. In some aspects wherein an rAAV vector also comprises a first transgene nucleic acid molecule and an at least second transgene nucleic acid molecule, the first promoter can be located upstream (5′) of the first transgene nucleic acid molecule and the at least second promoter can be located between the first transgene nucleic acid molecule and the at least second transgene nucleic acid molecule, such that the at least second promoter is downstream (3′) of the first transgene nucleic acid molecule and upstream (5′) of the at least second transgene nucleic acid molecule.

An rAAV vector can further comprise at least one enhancer. The at least one enhancer can be located anywhere in the rAAV vector. In some aspects, the at least one enhancer can be located immediately upstream (5′) of a promoter. Thus, an rAAV vector can comprise, in the 5′ to 3′ direction, a first AAV ITR sequence, an enhancer, a promoter sequence, a transgene nucleic acid molecule, a polyA sequence, and a second AAV ITR sequence. In some aspects, the at least one enhancer can be located immediately downstream (3′) of a promoter. Thus, an rAAV vector can comprise, in the 5′ to 3′ direction, a first AAV ITR sequence, a promoter sequence, an enhancer, a transgene nucleic acid molecule, a polyA sequence, and a second AAV ITR sequence. In some aspects, the at least one enhancer can be located immediately downstream of a transgene nucleic acid molecule. Thus, an rAAV vector can comprise, in the 5′ to 3′ direction, a first AAV ITR sequence, a promoter sequence, a transgene nucleic acid molecule, an enhancer, a polyA sequence, and a second AAV ITR sequence.

### AAV ITR Sequences

In some aspects, an AAV ITR sequence can comprise any AAV ITR sequence known in the art. In some aspects, an AAV ITR sequence can be an AAV1 ITR sequence, an AAV2 ITR sequence, an AAV4 ITR sequence, an AAV5 ITR sequence, an AAV6 ITR sequence, an AAV7 ITR sequence, an AAV8 ITR sequence, an AAV9 ITR sequence, an AAV10 ITR sequence, an AAV11 ITR sequence, an AAV12 ITR sequence, an AAV13 ITR sequence, an AAVrh74 ITR sequence or an AAVrh.10 ITR sequence.

Thus, in some aspects, an AAV ITR sequence can comprise, consist essentially of, or consist of an AAV1 ITR sequence, an AAV2 ITR sequence, an AAV4 ITR sequence, an AAV5 ITR sequence, an AAV6 ITR sequence, an AAV7 ITR sequence, an AAV8 ITR sequence, an AAV9 ITR sequence, an AAV10 ITR sequence, an AAV11 ITR sequence, an AAV12 ITR sequence, an AAV13 ITR sequence, an AAVrh74 ITR sequence, or an AAVrh.10 ITR sequence. In some embodiments, an AAV ITR sequence is a wildtype AAV ITR sequence. In some embodiments, an AAV ITR sequence is modified (e.g., mutated) AAV ITR sequence. In some embodiments, an rAAV vector described herein comprises one mutated AAV ITR and one wildtype AAV ITR.

In some aspects, an AAV ITR can comprise consist essentially of, or consist of a nucleic acid sequence at least 65%, 70%, 75%, 80%, 85%, 90%, 95%, 96%, 97%, 98%, 99% or 100% (or any percentage in between) identical to any AAV ITR nucleic acid sequence.

In some aspects, an rAAV provided herein comprises a first and a second AAV ITR sequence, wherein the first AAV ITR sequence comprises, consists essentially of, or consists of a nucleic acid sequence at least 65%, 70%, 75%, 80%, 85%, 90%, 95%, 96%, 97%, 98%, 99% or 100% (or any percentage in between) identical to any AAV ITR nucleic acid sequence and the second AAV ITR sequence comprises, consists essentially of, or consists of a nucleic acid sequence at least 65%, 70%, 75%, 80%, 85%, 90%, 95%, 96%, 97%, 98%, 99% or 100% (or any percentage in between) identical to any AVV ITR nucleic acid sequence.

### Promoter Sequences and Enhancers

A promoter or promoter sequence is a control sequence that is a region of a polynucleotide sequence at which the initiation and rate of transcription of a coding sequence, such as a gene or a transgene, are controlled. Promoters can be constitutive, inducible, repressible, or tissue-specific, for example. Promoters can contain genetic elements at which regulatory proteins and molecules such as RNA polymerase and transcription factors can bind. Non-limiting exemplary promoters include CK8e promoter, Rous sarcoma virus (RSV), LTR promoter (optionally with the RSV enhancer), a cytomegalovirus (CMV) promoter (e.g., SEQ ID NO:7; CGTTACATAA CTTACGGTAA ATGGCCCGCC TGGCTGACCG CCCAACGACC CCCGCCCATT GACGTCAATA ATGACGTATG TTCCCATAGT AACGCCAATA GGGACTTTCC ATTGACGTCA ATGGGTGGAG TATTTACGGT AAACTGCCCA CTTGGCAGTA CATCAAGTGT ATCATATGCC AAGTACGCCC CCTATTGACG TCAATGACGG TAAATGGCCC GCCTGGCATT ATGCCCAGTA CATGACCTTA TGGGACTTTC CTACTTGGCA GTACATCTAC GTATTAGTCA TCGCTATTAC CATGGTGATG CGGTTTTGGC AGTACATCAA TGGGCGTGGA TAGCGGTTTG ACTCACGGGG ATTTCCAAGT CTCCACCCCA TTGACGTCAA TGGGAGTTTG TTTTGGCACC AAAATCAACG GGACTTTCCA AAATGTCGTA ACAACTCCGC CCCATTGACG CAAATGGGCG GTAGGCGTGT ACGGTGGGAG GTCTATATAA GCAGAGCT), an SV40 promoter, a dihydrofolate reductase promoter, a β-actin promoter, a phosphoglycerol kinase (PGK) promoter, a U6 promoter, a synapsin promoter, an H1 promoter, a ubiquitous chicken β-actin hybrid (CBh) promoter, or a small nuclear RNA (U1a or U1b) promoter.

Additional non-limiting exemplary promoters provided herein include, but are not limited to EFla, Ubc, human β-actin, CAG, TRE, Ac5, Polyhedrin, CaMKIIa, Gal1, TEF1, GDS, ADH1, Ubi, and α-1-antitrypsin (hAAT). Nucleotide sequences of such promoters can be modified to increase or decrease the efficiency of mRNA transcription. Synthetically-derived promoters can be used for ubiquitous or tissue specific expression. Furthermore, virus-derived promoters, some of which are noted above, can be useful in the methods disclosed herein, e.g., CMV, HIV, adenovirus, and AAV promoters. In some aspects, a promoter is used together with at least one enhancer to increase the transcription efficiency. Non-limiting examples of enhancers include an interstitial retinoid-binding protein (IRBP) enhancer, an RSV enhancer or a CMV enhancer.

In some aspects, a promoter sequence can comprise, consist essentially of, or consist of a Rous sarcoma virus (RSV) LTR promoter sequence (optionally with the RSV enhancer), a cytomegalovirus (CMV) promoter sequence, an SV40 promoter sequence, a dihydrofolate reductase promoter sequence, a JeT promoter sequence, a strong a β-actin promoter sequence, a phosphoglycerol kinase (PGK) promoter sequence, a U6 promoter sequence, synapsin promoter, an H1 promoter sequence, a ubiquitous chicken β-actin hybrid (CBh) promoter sequence, a small nuclear RNA (U1a or U1b) promoter sequence, a VMD2 promoter sequence, an mRho promoter sequence, an EFI promoter sequence, an EFla promoter sequence, a Ubc promoter sequence, a human β-actin promoter sequence, a CAG promoter sequence, a TRE promoter sequence, an Ac5 promoter sequence, a polyhedrin promoter sequence, a CaMKIIa promoter sequence, a Gal1 promoter sequence, a TEF1 promoter sequence, a GDS promoter sequence, an ADH1 promoter sequence, a Ubi promoter sequence, a MeP426 promoter, or an α-1-an-titrypsin (hAAT) promoter sequence.

An enhancer is a regulatory element that increases the expression of a target sequence. A promoter/enhancer is a polynucleotide that contains sequences capable of providing both promoter and enhancer functions. For example, the long terminal repeats of retroviruses contain both promoter and enhancer functions. The enhancer/promoter can be endogenous or exogenous or heterologous. An endogenous enhancer/promoter is one that is naturally linked with a given gene in the genome. An exogenous or heterologous enhancer/promoter is one that is placed in juxtaposition to a gene by means of genetic manipulation (i.e., molecular biological techniques) or synthetic techniques such that transcription of that gene is directed by the linked enhancer/promoter. Non-limiting examples of linked enhancer/promoter for use in the methods, compositions and constructs provided herein include a PDE promoter plus IRBP enhancer or a CMV enhancer plus Ula promoter. Enhancers can operate from a distance and irrespective of their orientation relative to the location of an endogenous or heterologous promoter. An enhancer operating at a distance from a promoter is operably linked to that promoter irrespective of its location in the vector or its orientation relative to the location of the promoter.

Operably linked refers to the expression of a gene (i.e., a transgene) that is under the control of a promoter with which it is spatially connected. A promoter can be positioned 5′ (upstream) or 3′ (down-stream) of a gene under its control. A promoter can be positioned 5′ (upstream) of a gene under its control. The distance between a promoter and a gene can be approximately the same as the distance between that promoter and the gene it controls in the gene from which the promoter is derived. Variation in the distance between a promoter and a gene can be accommodated without loss of promoter function.

Any muscle specific promoter or regulatory cassette can be used. For example, MCK, dMCK, tMCK, CK6, CK7 and its sub-types/derivatives containing the mck Small Intron-1 Enhancer (SIEa-CK7, SIEb-CK7, SIEc-CK7, SIEd-CK7, SIEe-CK7, SIEf-CK7, SIEg-CK7, SIEh-CK7, SIEi-CK7, SIEj-CK7, etc.), MHCK7, CK8 and its sub-types/derivatives (CK8e, CK8a, CK8b, CK8g, CK8d, CK8e, CK8L, CK8M, etc.), human a-skeletal actin (HSA or ACTA1), desmin (DES), MLC2v, cTnT, Spc5-12, SP-301, MH or Sk-CRM4/DES gene promoters can be used. Synthetic regulatory cassettes containing muscle gene control elements juxtaposed in different combinations or with other known promoters (e.g., MHCK7, CK8, etc.), including the 13 Syn regulatory cassettes (Syn1 to Syn13), can also be used. In some aspects, a promoter sequence can comprise, consist essentially of, or consist of the CK8e promoter sequence. The CK8e promoter sequence can comprise, consist essentially of, or consist of a nucleic acid sequence at least 65%, 70%, 75%, 80%, 85%, 90%, 95%, 96%, 97%, 98%, 99% or 100% (or any percentage in between) identical to the nucleic acid sequence set forth in SEQ ID NO:5.

In some aspects, a promoter sequence can comprise, consist essentially of, or consist of a hybrid chicken β-actin promoter sequence.

As would be appreciated by the skilled artisan, a hybrid chicken β-actin promoter sequence can comprise a CMV sequence, a chicken β-actin promoter sequence, a chicken β-actin exon 1 sequence, a chicken β-actin intron 1 sequence, a minute virus of mice (MVM) intron sequence, or any combination thereof. In some aspects, a hybrid chicken β-actin promoter sequence can comprise, in the 5′ to 3′ direction, a CMV sequence, a chicken β-actin promoter sequence, chicken β-actin exon 1 sequence, a chicken β-actin intron 1 sequence and a minute virus of mice (MVM) intron sequence.

### PolyA Sequences

In some aspects, a polyadenylation (polyA) sequence can comprise any polyA sequence known in the art. A polyA sequence can be a synthetic polyA sequence or a polyA sequence derived from a naturally occurring protein. Non-limiting examples of polyA sequences include, but are not limited to, a retinol dehydrogenase 1 (RDH1) polyA sequence, a bovine growth hormone (BGH) polyA sequence, an SV40 polyA sequence, a SPA49 polyA sequence, a sNRP-TK65 polyA sequence, a sNRP polyA sequence, or a TK65 polyA sequence.

Thus, a polyA sequence can comprise, consist essentially of, or consist of a retinol dehydrogenase 1 (RDH1) polyA sequence, a bovine growth hormone (BGH) polyA sequence, an SV40 polyA sequence, a SPA49 polyA sequence, a sNRP-TK65 polyA sequence, a sNRP polyA sequence, or a TK65 polyA sequence.

In some aspects, a polyA sequence can comprise, consist essentially of, or consist of a human growth hormone polyA sequence. In some aspects, a polyA sequence can comprise, consist essentially of, or consist of a nucleic acid sequence at least 65%, 70%, 75%, 80%, 85%, 90%, 95%, 96%, 97%, 98%, 99% or 100% (or any percentage in between) identical to the sequence set forth at 2569-3048 bp of SEQ ID NO:2.

In certain embodiments, an rAAV vector disclosed herein comprises a Kozak sequence. In some aspects, a Kozak sequence can comprise, consist essentially of, or consist of a nucleic acid sequence at least 65%, 70%, 75%, 80%, 85%, 90%, 95%, 96%, 97%, 98%, 99% or 100% (or any percentage in between) identical to the sequence set forth at 1193-1202 bp of SEQ ID NO:2.

In some aspects, an rAAV vector of the present disclosure can comprise, consist essentially of, or consist of a nucleic acid sequence at least 65%, 70%, 75%, 80%, 85%, 90%, 95%, 96%, 97%, 98%, 99% or 100% (or any percentage in between) identical to the sequence put forth in SEQ ID NO:2.

In some embodiments, an rAAV vector of the present disclosure consists of or comprises the sequence set forth in SEQ ID NO:2 with one or more (e.g., 1, 2, 3, 4, 5, 6, 7, 8, 9, 10, 11, 12, 13, 14, 15, 16, 17, 18, 19, or 20) conservative amino acid substitutions.

In certain embodiments, an rAAV vector described herein comprises, in 5′ to 3′ order, a first AAV2 ITR of bp 1-141 of SEQ ID NO:2; a CK8e promoter of SEQ ID NO:5; a codon optimized transgene encoding human Smad7 of SEQ ID NO:1; a polyA sequence of 2569-3048 bp of SEQ ID NO: 2; and a second AAV2 ITR of bp 3087-3228 of SEQ ID NO:2.

### Bacterial Plasmids

In some aspects, the rAAV vectors of the present disclosure can be contained within a bacterial plasmid to allow for propagation of the rAAV vector in vitro. Thus, the present disclosure provides bacterial plasmids comprising any of the rAAV vectors described herein. A bacterial plasmid can further comprise an origin of replication sequence. A bacterial plasmid can further comprise an antibiotic resistance gene. A bacterial plasmid can further comprise a resistance gene promoter. A bacterial plasmid can further comprise a prokaryotic promoter. In some aspects, a bacterial plasmid of the present disclosure can comprise, consist essentially of, or consist of a nucleic acid sequence at least 65%, 70%, 75%, 80%, 85%, 90%, 95%, 96%, 97%, 98%, 99% or 100% (or any percentage in between) identical to any of the nucleic acid sequence set forth in SEQ ID NO:2.

### Origin of Replication Sequence

In some aspects, an origin of replication sequence can comprise, consist essentially of, or consist of any origin of replication sequence known in the art. An origin of replication sequence can be a bacterial origin of replication sequence, thereby allowing the rAAV vector comprising the bacterial origin of replication sequence to be produced, propagated, and maintained in bacteria, using methods standard in the art.

### Antibiotic Resistance Genes

In some aspects, bacterial plasmids, rAAV vectors and/or rAAV viral vectors of the disclosure can comprise an antibiotic resistance gene.

In some aspects, an antibiotic resistance gene can comprise, consist essentially of, or consist of any antibiotic resistance genes known in the art. Examples of antibiotic resistance genes known in the art include, but are not limited to kanamycin resistance genes, spectinomycin resistance genes, streptomycin resistance genes, ampicillin resistance genes, carbenicillin resistance genes, bleomycin resistance genes, erythromycin resistance genes, polymyxin B resistance genes, tetracycline resistance genes and chloramphenicol resistance genes.

In some aspects, an antibiotic resistance gene can be any suitable resistance gene such as a kanamycin or ampicillin resistance gene.

### Viral Vector

A viral vector is a recombinantly produced virus or viral particle that contains a polynucleotide to be delivered into a host cell, either in vivo, ex vivo or in vitro. Examples of viral vectors include retroviral vectors, AAV vectors, lentiviral vectors, adenovirus vectors, alphavirus vectors and the like. Alphavirus vectors, such as Semliki Forest virus-based vectors and Sindbis virus-based vectors, have also been developed for use in gene therapy and immunotherapy.

An AAV virion, AAV viral particle, AAV viral vector, rAAV viral vector, AAV vector particle, or AAV particle is a viral particle composed of at least one AAV capsid protein and an encapsidated polynucleotide rAAV vector. Thus, production of an rAAV viral vector necessarily includes production of an rAAV vector, as such a vector is contained within an rAAV vector.

A viral capsid or capsid refers to the proteinaceous shell or coat of a viral particle. Capsids function to encapsidate, protect, transport, and release into the host cell a viral genome. Capsids are generally comprised of oligomeric structural subunits of protein (“capsid proteins”). Encapsidated means enclosed within a viral capsid. A viral capsid of AAV is composed of a mixture of three viral capsid proteins: VP1, VP2, and VP3. The mixture of VP1, VP2 and VP3 contains 60 monomers that are arranged in a T=1 icosahedral symmetry in a ratio of 1:1:10 (VP1:VP2:VP3) or 1:1:20 (VP1:VP2:VP3).

The present disclosure provides an rAAV viral vector comprising: a) any of the rAAV vectors described herein, or complement thereof; and b) an AAV capsid protein.

The present disclosure provides an rAAV viral vector comprising: a) any of the rAAV vectors described herein; and b) an AAV capsid protein.

An AAV capsid protein can be any AAV capsid protein known in the art. An AAV capsid protein can be an AAV1 capsid protein, an AAV2 capsid protein, an AAV4 capsid protein, an AAV5 capsid protein, an AAV6 capsid protein, an AAV7 capsid protein, an AAV8 capsid protein, an AAV9 capsid protein, an AAV10 capsid protein, an AAV11 capsid protein, an AAV12 capsid protein, an AAV13 capsid protein, an AAVPHP.B capsid protein, an AAVrh74 capsid protein, an AAVrh.10 capsid protein or any muscle-specific/trophic AAV capsid protein derived from the genetic engineering or directed evolution of the capsids listed here, including but not limited to MyoAAV 1A-1F and MyoAAV 2A.

### Compositions and Pharmaceutical Compositions

The present disclosure provides compositions comprising any of the isolated polynucleotides, rAAV vectors, and/or rAAV viral vectors described herein. In some aspects, the compositions can be pharmaceutical compositions. Accordingly, the present disclosure provides pharmaceutical compositions comprising any of the isolated polynucleotides, rAAV vectors, and/or rAAV viral vectors described herein.

The pharmaceutical composition, as described herein, can be formulated by any methods known or developed in the art of pharmacology, which include but are not limited to contacting the active ingredients (e.g., viral particles or recombinant vectors) with an excipient and/or additive and/or other accessory ingredient, dividing or packaging the product to a dose unit. The viral particles of this disclosure can be formulated with desirable features, e.g., increased stability, increased cell transfection, sustained or delayed release, biodistributions or tropisms, modulated or enhanced translation of encoded protein in vivo, and the release profile of encoded protein in vivo.

As such, the pharmaceutical composition can further comprise saline, lipidoids, liposomes, lipid nanoparticles, polymers, lipoplexes, core-shell nanoparticles, peptides, proteins, cells transfected with viral vectors (e.g., for transplantation into a subject), nanoparticle mimics or combinations thereof. In some aspects, the pharmaceutical composition is formulated as a nanoparticle. In some aspects, the nanoparticle is a self-assembled nucleic acid nanoparticle.

A pharmaceutical composition in accordance with the present disclosure can be prepared, packaged, and/or sold in bulk, as a single unit dose, and/or as a plurality of single unit doses. The amount of the active ingredient is generally equal to the dosage of the active ingredient which would be administered to a subject and/or a convenient fraction of such a dosage such as, for example, one-half or one-third of such a dosage. The formulations can include one or more excipients and/or additives, each in an amount that together increases the stability of the viral vector, increases cell transfection or transduction by the viral vector, increases the expression of viral vector encoded protein, and/or alters the release profile of viral vector encoded proteins. In some aspects, the pharmaceutical composition comprises an excipient and/or additive. Non limiting examples of excipients and/or additives include solvents, dispersion media, diluents, or other liquid vehicles, dispersion or suspension aids, surface active agents, isotonic agents, thickening or emulsifying agents, preservatives, or combination thereof.

In some aspects, a pharmaceutical composition comprises a cryoprotectant, which is an agent capable of reducing or eliminating damage to a substance during freezing. Non-limiting examples of cryoprotectants include sucrose, trehalose, lactose, glycerol, dextrose, raffinose and/or mannitol.

A pharmaceutically acceptable carrier encompasses any standard pharmaceutical carrier, such as a phosphate buffered saline solution, water, and emulsions, such as an oil/water or water/oil emulsion, and various types of wetting agents. The compositions also can include stabilizers and preservatives. For examples of carriers, stabilizers and adjuvants, see Martin (1975) Remington's Pharm. Sci., 15th Ed. (Mack Publ. Co., Easton).

In some aspects, a pharmaceutical composition can comprise phosphate-buffered saline (PBS), D-sorbitol or any combination thereof.

In some aspects, a pharmaceutical composition can comprise PBS, wherein the PBS is present at a concentration of about 100 mM to about 500 mM, or about 200 mM to about 400 mM, or about 300 mM to about 400 mM. In some aspects, sodium chloride can be present at a concentration of about 350 mM.

In some aspects, a pharmaceutical composition can comprise D-sorbitol, wherein the D-sorbitol is present at a concentration of about 1% to about 10%, or about 2.5% to about 7.5%. In some aspects, the D-sorbitol can be present at a concentration of about 5%.

Thus, the present disclosure provides a pharmaceutical composition comprising an rAAV vector and/or rAAV viral vector in a 350 mM phosphate-buffered saline solution comprising D-sorbitol at a concentration of 5%.

### Methods of Using Compositions

The present disclosure provides the use of a disclosed composition or pharmaceutical composition for the treatment of a disease or disorder in a cell, tissue, organ, animal, or subject, as known in the art or as described herein, using the disclosed compositions and pharmaceutical compositions, e.g., administering or contacting the cell, tissue, organ, animal, or subject with a therapeutic effective amount of the composition or pharmaceutical composition. In one aspect, the subject is a mammal. The subject can be a human.

This disclosure provides methods of preventing or treating a disease and/or disorder, comprising, consisting essentially of, or consisting of administering to a subject a therapeutically effective amount of any one of the rAAV vectors, rAAV viral vectors, compositions and/or pharmaceutical compositions disclosed herein.

In some aspects, the disease and/or disorder can be a genetic disorder involving the Smad7 gene. A genetic disorder involving a Smad7 gene can involve dystrophic muscle. Genetic disorders involving the Smad7 gene include, but are not limited to, obesity-related disorders, include sarcopenic obesity and type 2 diabetes mellitus. Methods described herein can also be used to enhance muscle mass and function in patients without a Smad7 gene disorder.

A disease or disorder can be a muscle wasting disease or condition such as occurs with cancer; a state of pronounced weight loss frailty and fatigue; sarcopenia; heart failure; chronic obstructive pulmonary disease (COPD); end-stage renal disease; chronic infection; hip fracture; malnutrition and burns and sepsis; muscular dystrophies, myopathies, inclusion body myositis (sporadic and hereditary), polymyositis, dermatomyositis, necrotizing autoimmune myopathy, and neuromuscular diseases. In some aspects, the subject does not have a disorder or disease associated with muscle wasting.

In some aspects, the disease can be a disorder involving the Smad7 protein. A genetic disorder involving the Smad7 protein can be Smad7 loss, misfunction and/or deficiency.

In some aspects, a disease can be a disease that is characterized by the loss-of-function of at least one copy of a Smad7 gene in the genome of a subject. In some aspects, a disease can be a disease that is characterized by a decrease in function of at least one copy of the Smad7 gene in the genome of a subject. In some aspects, a disease can be a disease that is characterized by at least one mutation in at least one mutation in at least one copy of the Smad7 gene in the genome of the subject.

A subject in the methods provided herein can be deficient in Smad7 and/or Smad7. As used herein, “Smad7 deficiency” means that a subject can have one or more mutations in a Smad7 gene or lacks a functional Smad7 gene. As used herein, “Smad7 deficiency” means that a subject can have one or more mutations in the Smad7 protein or lacks a functional Smad7 protein. Optionally, a subject is not deficient in Smad7 and/or Smad7.

A mutation in an Smad7 gene or Smad7 protein can be any type of mutation that is known in the art. Non-limiting examples of mutations include somatic mutations, single nucleotide variants (SNVs), nonsense mutations, insertions, deletions, duplications, frameshift mutations, repeat expansions, short insertions and deletions (INDELs), long INDELs, alternative splicing, the products of alternative splicing, altered initiation of translation, the products of altered initiation of translation, proteomic cleavage, the products of proteomic cleavage.

In some aspects, a disease can be a disease that is characterized by a decrease in expression of Smad7 gene in a subject as compared to a control subject that does not have the disease. In some aspects, the decrease in expression can be at least about 10%, or at least about 20%, or at least about 30%, or at least about 40%, or at least about 50%, or at least about 60%, or at least about 70%, or at least about 80%, or at least about 90%, or at least about 95%, or at least about 99%, or at least about 100%.

In some aspects, a disease can be a disease that is characterized by a decrease in the amount of Smad7 protein in a subject as compared to a control subject that does not have the disease. In some aspects, the decrease in the amount of Smad7 protein can be at least about 10%, or at least about 20%, or at least about 30%, or at least about 40%, or at least about 50%, or at least about 60%, or at least about 70%, or at least about 80%, or at least about 90%, or at least about 95%, or at least about 99%, or at least about 100%.

In some aspects, a disease can be a disease that is characterized by a decrease in the activity of Smad7 protein in a subject as compared to a control subject that does not have the disease. In some aspects, the decrease in the activity of Smad7 protein can be at least about 10%, or at least about 20%, or at least about 30%, or at least about 40%, or at least about 50%, or at least about 60%, or at least about 70%, or at least about 80%, or at least about 90%, or at least about 95%, or at least about 99%, or at least about 100%.

Methods of treatment can alleviate one or more symptoms of a disease and/or disorder described herein. In an embodiment, delivery of compositions described herein can prevent or delay development of detectable symptoms, if administered to a subject carrying a mutation in the Smad7 gene before symptoms become detectable. Therefore, treatment can be therapeutic or prophylactic. Therapy refers to inhibition or reversal of established symptoms or phenotype. Therapy can also mean delay of onset of symptoms or phenotype. Prophylaxis means inhibiting or preventing development of symptoms in subjects not already displaying overt symptoms. Subjects not displaying overt symptoms can be identified early in life as carrying a loss of function mutation in a Smad7 gene by appropriate genetic testing performed before 18 months, 12 months, or 6 months of age.

In some aspects, treatment according to the methods described herein can increase striated muscle (skeletal and cardiac) mass, can increase striated muscle function, can increase striated muscle fiber size, can increase striated muscle force, can increase mass of dystrophic striated muscle, can increase function of dystrophic striated muscle, can increase fiber size of dystrophic striated muscle, can increase the force of dystrophic striated muscle, and/or can repress ActRIIB-mediated atrophic signaling in striated muscle.

A subject to be treated using the methods, compositions, pharmaceutical compositions, rAAV vectors or rAAV viral vectors of the present disclosure can have any of the diseases and/or symptoms described herein.

In some aspects, a subject can be less than 0.5 years of age, or less than 1 year of age, or less than 1.5 years of age, or less than 2 years of age, or at less than 2.5 years of age, or less than 3 years of age, or less than 3.5 years of age, or less than 3.5 years of age, or less than 4 years of age, or less than 4.5 years of age, or less than 5 years of age, or less than 5.5 years of age, or less than 6 years of age, or less than 6.5 years of age, or less than 7 years of age, or less than 7.5 years of age, or less than 8 years of age, or less than 8.5 years of age, or less than 9 years of age, or less than 9.5 years of age, or less than 10 years of age. In some aspects the subject can be less than 11 years of age, less than 12 years of age, less than 13 years of age, less than 14 years of age, less than 15 years of age, less than 20 years of age, less than 30 years of age, less than 40 years of age, less than 50 years of age, less than 60 years of age, less than 70 years of age, less than 80 years of age, less than 90 years of age, less than 100 years of age, less than 110 years of age, or less than 120 years of age. In some aspects, a subject can be less than 0.5years of age. In some aspects, a subject can be less than 4 years of age. In some aspects, a subject can be less than 10 years of age.

The methods of treatment and prevention disclosed herein may be combined with appropriate diagnostic techniques to identify and select patients for the therapy or prevention.

The disclosure provides methods of increasing the level of a protein in a host cell, comprising contacting the host cell with any one of the rAAV viral vectors disclosed herein, wherein the rAAV viral vectors comprises any one of the rAAV vectors disclosed herein, comprising a transgene nucleic acid molecule encoding the protein. In some aspects, the protein is a therapeutic protein. In some aspects, the host cell is in vitro, in vivo, or ex vivo. In some aspects, the host cell is derived from a subject. In some aspects, the subject suffers from a disorder, which results in a reduced level and/or functionality of the protein, as compared to the level and/or functionality of the protein in a normal subject.

In some aspects, the level of the protein is increased to level of about 1×10−7 ng, about 3×10−7 ng, about 5×10−7 ng, about 7×10−7 ng, about 9×10−7 ng, about 1×10−6 ng, about 2×10−6 ng, about 3×10−6 ng, about 4×10−6 ng, about 6×10−6 ng, about 7×10−6 ng, about 8×10−6 ng, about 9×10−6 ng, about 10×10−6 ng, about 12×10−6 ng, about 14×10−6 ng, about 16×10−6 ng, about 18×10−6 ng, about 20×10−6 ng, about 25×10−6 ng, about 30×10−6 ng, about 35×10−6 ng, about 40×10−6 ng, about 45×10−6 ng, about 50×10−6 ng, about 55×10−6 ng, about 60×10−6 ng, about 65×10−6 ng, about 70×10−6 ng, about 75×10−6 ng, about 80×10−6 ng, about 85×10−6 ng, about 90×10−6 ng, about 95×10−6 ng, about 10×10−5 ng, about 20×10−5 ng, about 30×10−5 ng, about 40×10−5 ng, about 50×10−5 ng, about 60×10−5 ng, about 70×10−5 ng, about 80×10−5 ng, or about 90×10−5 ng in the host cell.

The expression levels of a gene (e.g., Smad7) or a protein (e.g., Smad7) can be determined by any suitable method known in the art or described herein. Protein levels may be determined, for example, by western Blotting, immunohistochemistry and flow cytometry. Gene expression can be determined, for example, by quantitative PCR, gene sequencing, and RNA sequencing.

The disclosure provides methods of introducing a gene of interest to a cell in a subject comprising contacting the cell with an effective amount of any one of the rAAV viral vectors disclosed herein, wherein the rAAV viral vectors contain any one of the rAAV vectors disclosed herein, comprising the gene of interest.

In some aspects of the methods of the present disclosure, a subject can also be administered a prophylactic immunosuppressant treatment regimen in addition to being administered an rAAV vector or rAAV viral vector of the present disclosure. In some aspects, an immunosuppressant treatment regimen can comprise administering at least one immunosuppressive therapeutic. Non limiting examples of immunosuppressive therapeutics include, but are not limited to, Sirolimus (rapamycin), acetaminophen, diphenhydramine, IV methylprednisolone, prednisone, or any combination thereof. An immunosuppressive therapeutic can be administered prior to the day of administration of the rAAV vector and/or rAAV viral vector, on the same day as the administration of the rAAV vector and/or rAAV viral vector, or any day following the administration of the rAAV vector and/or rAAV viral vector.

A “subject” of diagnosis or treatment is a cell or an animal such as a mammal, or a human. The terms “subject” and “patient” are used interchangeably herein. A subject is not limited to a specific species and includes non-human animals subject to diagnosis or treatment and those subject to infections or animal models, including, without limitation, simian, murine, rat, canine, or leporid species, as well as other livestock, sport animals, or pets. In some aspects, the subject is a human. In some embodiments, the subject is a human child, e.g., a child of less than five years of age. In some embodiments, the subject is a human newborn, e.g., a newborn of less than one month, less than two months, less than three months, or less than four months of age.

As used herein, “treating” or “treatment” of a disease in a subject refers to (1) inhibiting the disease or arresting its development; or (2) ameliorating or causing regression of the disease or the symptoms of the disease. As understood in the art, “treatment” is an approach for obtaining beneficial or desired results, including clinical results. For the purposes of the present technology, beneficial or desired results can include one or more, but are not limited to, alleviation or amelioration of one or more symptoms, diminishment of extent of a condition (including a disease), stabilized (i.e., not worsening) state of a condition (including disease), delay or slowing of condition (including disease), progression, amelioration or palliation of the condition (including disease), states and remission (whether partial or total), whether detectable or undetectable.

As used herein, “preventing” or “prevention” of a disease refers to preventing the symptoms or disease from occurring in a subject that is predisposed or does not yet display symptoms of the disease.

As used herein the term “effective amount” intends to mean a quantity sufficient to achieve a desired effect. In the context of therapeutic or prophylactic applications, the effective amount will depend on the type and severity of the condition at issue and the characteristics of the individual subject, such as general health, age, sex, body weight, and tolerance to pharmaceutical compositions. In the context of gene therapy, the effective amount can be the amount sufficient to result in regaining part or full function of a gene that is deficient in a subject. In some aspects, the effective amount of an rAAV viral vector is the amount sufficient to result in expression of a gene in a subject such that a Smad7 polypeptide is produced. In some aspects, the effective amount is the amount required to increase striated muscle mass and/or function in subject by at least 10%, at least 20%, at least 30%, at least 40%, at least 50%, at least 60%, at least 70%, at least 80%, or at least 90% compared to a subject who has not been administered an rAAV viral vector described herein or has been administered a control treatment. The skilled artisan will be able to determine appropriate amounts depending on these and other factors.

In some aspects, the effective amount will depend on the size and nature of the application in question. It will also depend on the nature and sensitivity of the target subject and the methods in use. The skilled artisan will be able to determine the effective amount based on these and other considerations. The effective amount may comprise, consist essentially of, or consist of one or more administrations of a composition depending on the embodiment.

As used herein, the term “administer” or “administration” intends to mean delivery of a substance to a subject such as an animal or human. Administration can be effected in one dose, continuously or intermittently throughout the course of treatment. Methods of determining the most effective means and dosage of administration are known to those of skill in the art and will vary with the composition used for therapy, the purpose of the therapy, as well as the age, health or gender of the subject being treated. Single or multiple administrations can be carried out with the dose level and pattern being selected by the treating physician or in the case of pets and other animals, treating veterinarian.

Methods of determining the most effective means and dosage of administration are known to those of skill in the art and will vary with the composition used for therapy, the purpose of the therapy and the subject being treated. Single or multiple administrations can be carried out with the dose level and pattern being selected by the treating physician. It is noted that dosage may be impacted by the route of administration. Suitable dosage formulations and methods of administering the agents are known in the art. Non-limiting examples of such suitable dosages may be as low as 109 vector genomes to as much as 1017 vector genomes per administration.

In some aspects of the methods described herein, the number of viral particles (e.g., rAAV viral vectors) administered to the subject ranges from about 109 to about 1017. In some aspects, about 1010to about 1012, about 1011 to about 1013, about 1011 to about 1012, about 1011 to about 1014, about 1012 to about 1016, about 1013 to about 1016, about 1014 to about 1015, about 5×1011 to about 5×1012, about 1011 to about 1018, about 1013 to about 1016, or about 1012 to about 1013 viral particles are administered to the subject.

In some aspects of the methods described herein, the number of viral particles (e.g., rAAV viral vectors) administered to the subject is at least about 1010, or at least about 1011, or at least about 1012, or at least about 1013, or at least about 1014, or at least about 1015, or at least about 1016, or at least about 1017 viral particles.

In some aspects of the methods described herein, the dosing (vector genomes/kg body mass or vg/kg) and the number of total viral particles administered to the subject can depend on the age and mass of the subject. In non-limiting examples, a subject about 8 years of age weighing 25 kg and administered 1×1014 vg/kg will receive about 2.5×1015 viral particles whereas a subject about 50 years of age weighing 80 kg will receive about 8×1015 viral particles with this dose. In another non-limiting example, the dosing may be adjusted for a younger subject, as with a subject about 1 year of age weighing 10 kg and administered 1×1013 vg/kg to receive 1×1014 viral particles.

In some aspects, the amounts of viral particles in a composition, pharmaceutical composition, or the amount of viral particles administered to a patient can be calculated based on the percentage of viral particles that are predicted to contain viral genomes.

In some aspects, rAAV viral vectors of the present disclosure can be introduced to the subject intravenously, intrathecally (IT), intracisterna-magna (ICM) intracerebrally, intraventricularly, intranasally, intratracheally, intra-aurally, intra-ocularly, or peri-ocularly, orally, rectally, transmucosally, inhalationally, transdermally, parenterally, subcutaneously, intradermally, intramuscularly, intracisternally, intranervally, intrapleurally, topically, intralymphatically, intracisternally; such introduction may also be intra-arterial, intracardiac, subventricular, epidural, intracerebral, intracerebroventricular, subretinal, intravitreal, intraarticular, intraperitoneal, intrauterine, intranerve or any combination thereof. In some aspects, the viral particles are delivered to a desired target tissue, e.g., to muscle, nervous system, as non-limiting examples. In some aspects, delivery of viral particles is systemic. In some aspects, rAAV viral vectors of the present disclosure are administered intrathecally (IT). In some aspects, rAAV viral vectors of the present disclosure are administered intracisterna-manga (ICM).

In some aspects, the rAAV viral vectors of the present disclosure repair a gene deficiency in a subject. In some aspects, the ratio of repaired target polynucleotide or polypeptide to unrepaired target polynucleotide or polypeptide in a successfully treated cell, tissue, organ or subject is at least about 1.5:1, about 2:1, about 3:1, about 4:1, about 5:1, about 6:1, about 7:1, about 8:1, about 9:1, about 10:1, about 20:1, about 50:1, about 100:1, about 1000:1, about 10,000:1, about 100,000:1, or about 1,000,000:1. The amount or ratio of repaired target polynucleotide or polypeptide can be determined by any method known in the art, including but not limited to western blot, northern blot, Southern blot, PCR, sequencing, mass spectrometry, flow cytometry, immunohistochemistry, immunofluorescence, fluorescence in situ hybridization, next generation sequencing, immunoblot, and ELISA.

Administration of the rAAV vectors, rAAV viral vectors, compositions or pharmaceutical compositions of this disclosure can be affected in one dose, continuously or intermittently throughout the course of treatment. In some aspects, the rAAV vectors, rAAV viral vectors, compositions, or pharmaceutical compositions of this disclosure are parenterally administered by injection, infusion, or implantation.

In some aspects, the rAAV viral vectors of this disclosure show enhanced tropism for skeletal and/or cardiac muscle.

In some embodiments, the subject is administered one single dose of a recombinant rAAV provided herein in its lifetime. In some embodiments, a subject is administered repeat doses of the recombinant rAAV provided herein. These repeat doses may contain the same amount of rAAV particles or they can contain different amounts of rAAV particles. In some embodiments, the subject is administered repeat doses of the rAAV about every 6 months, about every 9 months, about every 12 months, about every 15 months, about every 18 months, about every 2 years, about every 3 years, about every 4 years, about every 5 years, about every 6 years, about every 7 years, about every 8 years, about every 9 years, or about every 10 years.

### Methods of Manufacture

A variety of approaches can be used to produce rAAV viral vectors of the present disclosure. In some aspects, packaging is achieved by using a helper virus or helper plasmid and a cell line. The helper virus or helper plasmid contains elements and sequences that facilitate viral vector production. In another aspect, the helper plasmid is stably incorporated into the genome of a packaging cell line, such that the packaging cell line does not require additional transfection with a helper plasmid.

In some aspects, the cell is a packaging or helper cell line. In some aspects, the helper cell line is eukaryotic cell; for example, an HEK 293 cell or 293T cell. In some aspects, the helper cell is a yeast cell or an insect cell.

In some aspects, the cell comprises a nucleic acid encoding a tetracycline activator protein; and a promoter that regulates expression of the tetracycline activator protein. In some aspects, the promoter that regulates expression of the tetracycline activator protein is a constitutive promoter. In some aspects, the promoter is a phosphoglycerate kinase promoter (PGK) or a CMV promoter.

A helper plasmid can comprise, for example, at least one viral helper DNA sequence derived from a replication-incompetent viral genome encoding in trans all virion proteins required to package a replication incompetent AAV, and for producing virion proteins capable of packaging the replication-incompetent AAV at high titer, without the production of replication-competent AAV.

Helper plasmids for packaging AAV are disclosed in, for example, U.S. Patent Pub. No. 2004/0235174 A1, incorporated herein by reference. As stated therein, an AAV helper plasmid can contain as helper virus DNA sequences, by way of non-limiting example, the Ad5 genes E2A, E4 and VA, controlled by their respective original promoters or by heterologous promoters. AAV helper plasmids can additionally contain an expression cassette for the expression of a marker protein such as a fluorescent protein to permit the simple detection of transfection of a desired target cell.

The disclosure provides methods of producing rAAV viral vectors comprising transfecting a packaging cell line with any one of the AAV helper plasmids disclosed herein; and any one of the rAAV vectors disclosed herein. In some aspects, the AAV helper plasmid and rAAV vector are co-transfected into the packaging cell line. In some aspects, the cell line is a mammalian cell line, for example, human embryonic kidney (HEK) 293 cell line. The disclosure provides cells comprising any one of the rAAV vectors and/or rAAV viral vectors disclosed herein.

As used herein, the term “helper” in reference to a virus or plasmid refers to a virus or plasmid used to provide the additional components necessary for replication and packaging of any one of the rAAV vectors disclosed herein. The components encoded by a helper virus can include any genes required for virion assembly, encapsidation, genome replication, and/or packaging. For example, the helper virus or plasmid may encode necessary enzymes for the replication of the viral genome. Non-limiting examples of helper viruses and plasmids suitable for use with AAV constructs include pHELP (plasmid), adenovirus (virus), or herpesvirus (virus). In some aspects, the pHELP plasmid may be the pHELPK plasmid, wherein the ampicillin expression cassette is exchanged with a kanamycin expression cassette.

As used herein, a packaging cell (or a helper cell) is a cell used to produce viral vectors. Producing recombinant AAV viral vectors can require Rep and Cap proteins provided in trans as well as gene sequences from Adenovirus that help AAV replicate. In some aspects, packaging/helper cells contain a plasmid that is stably incorporated into the genome of the cell. In other aspects, the packaging cell can be transiently transfected. Typically, a packaging cell is a eukaryotic cell, such as a mammalian cell or an insect cell.

The compositions and methods are more particularly described below and the Examples set forth herein are intended as illustrative only, as numerous modifications and variations therein will be apparent to those skilled in the art. The terms used in the specification generally have their ordinary meanings in the art, within the context of the compositions and methods described herein, and in the specific context where each term is used. Some terms have been more specifically defined herein to provide additional guidance to the practitioner regarding the description of the compositions and methods.

### Definitions and Incorporations

As used herein, the term “and/or” includes any and all combinations of one or more of the associated listed items. As used in the description herein and throughout the claims that follow, the meaning of “a”, “an”, and “the” includes plural reference as well as the singular reference unless the context clearly dictates otherwise. The term “about” in association with a numerical value means that the value varies up or down by 5%. For example, for a value of about 100, means 95 to 105 (or any value between 95 and 105).

All patents, patent applications, and other scientific or technical writings referred to anywhere herein are incorporated by reference herein in their entirety. The embodiments illustratively described herein suitably can be practiced in the absence of any element or elements, limitation or limitations that are specifically or not specifically disclosed herein. Thus, for example, in each instance herein any of the terms “comprising,” “consisting essentially of,” and “consisting of” can be replaced with either of the other two terms, while retaining their ordinary meanings. The terms and expressions which have been employed are used as terms of description and not of limitation, and there is no intention that in the use of such terms and expressions of excluding any equivalents of the features shown and described or portions thereof, but it is recognized that various modifications are possible within the scope of the claims. Thus, it should be understood that although the present methods and compositions have been specifically disclosed by embodiments and optional features, modifications and variations of the concepts herein disclosed can be resorted to by those skilled in the art, and that such modifications and variations are considered to be within the scope of the compositions and methods as defined by the description and the appended claims.

Any single term, single element, single phrase, group of terms, group of phrases, or group of elements described herein can each be specifically excluded from the claims.

Whenever a range is given in the specification, for example, a temperature range, a time range, a composition, or concentration range, all intermediate ranges and subranges, as well as all individual values included in the ranges given are intended to be included in the disclosure. It will be understood that any subranges or individual values in a range or subrange that are included in the description herein can be excluded from the aspects herein. It will be understood that any elements or steps that are included in the description herein can be excluded from the claimed compositions or methods

In addition, where features or aspects of the compositions and methods are described in terms of Markush groups or other grouping of alternatives, those skilled in the art will recognize that the compositions and methods are also thereby described in terms of any individual member or subgroup of members of the Markush group or other group.

The following are provided for exemplification purposes only and are not intended to limit the scope of the embodiments described in broad terms above.

### Example 1: Materials and Methods

Antibodies. Antibodies against Smad7 and GAPDH were obtained from Abcam (Boston, MA) whereas fluorescent secondary antibodies were obtained from LI-COR (Lincoln, NB).

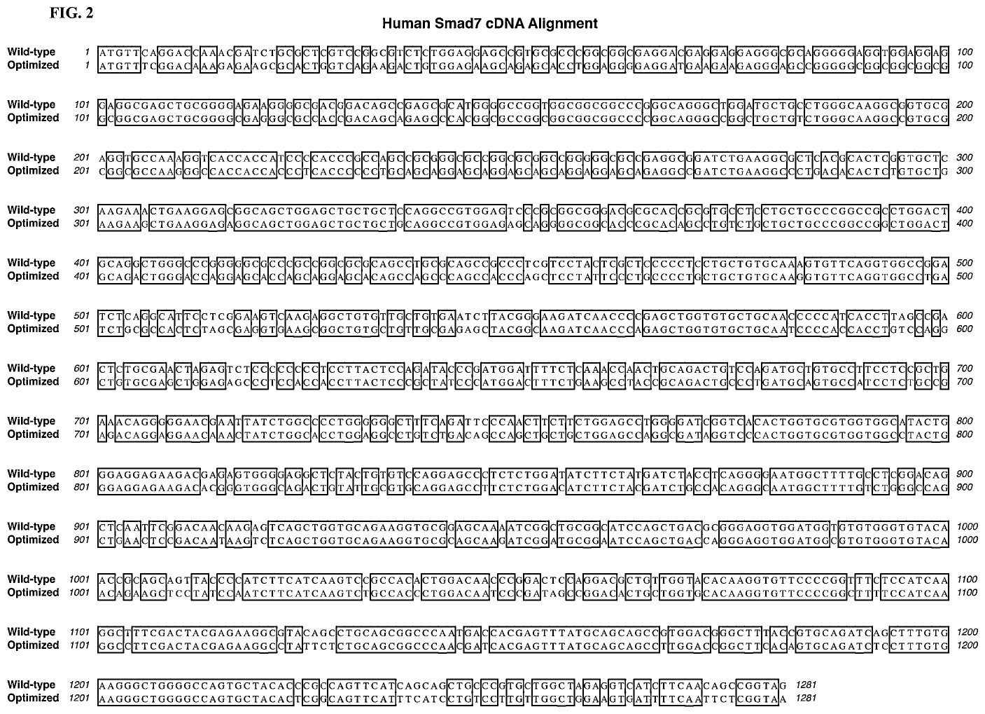

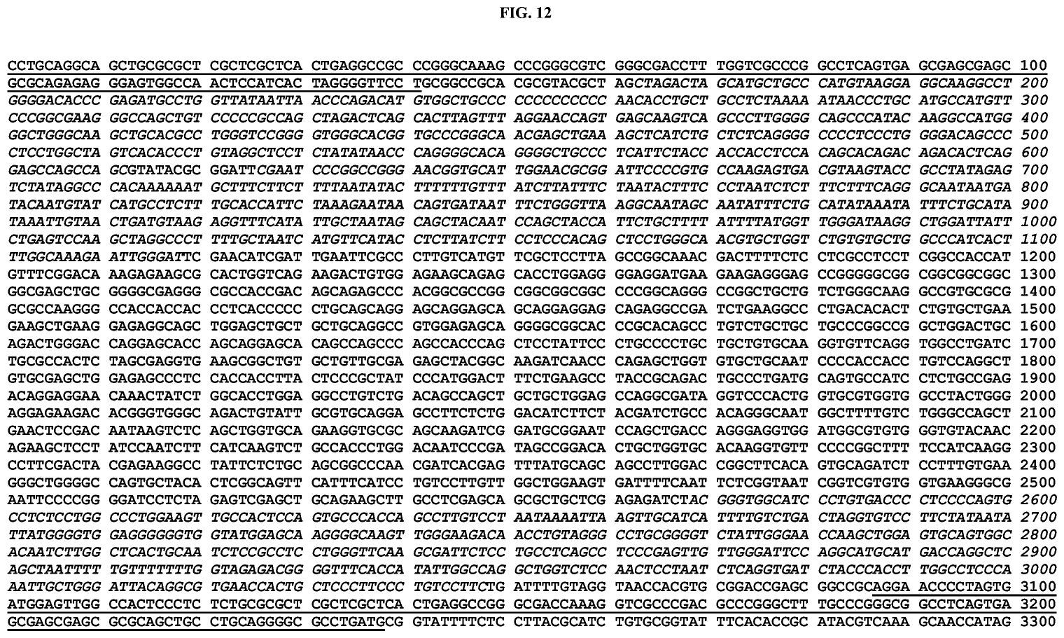

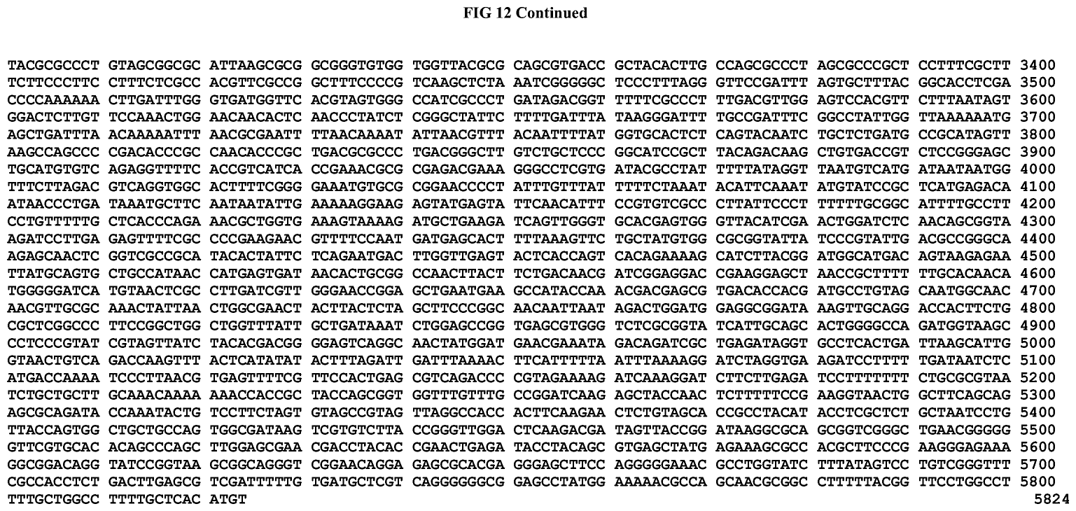

Production of rAAV6 vectors. CMV and CK8 gene promoters as well as cDNA constructs encoding wild-type mSmad7 (GenBank Reference #NM_001042660.1) or codon-optimized hSmad7 (see SEQ ID NO: 1 and FIG. 2) were synthesized (GenScript, Piscataway, NJ) and cloned into rAAV expression plasmids containing an hGH poly-A region, all of which were flanked by AAV2 inverted terminal repeats (see SEQ ID NO: 2, FIG. 11 and FIG. 12). These plasmids were then used to manufacture recombinant vectors in the Research Vector Core, Children's Hospital of Philadelphia. Each was transfected with the pDGM6 packaging plasmid containing the AAV6 cap genes, AAV2 rep genes and adenovirus helper genes into HEK293 cells to generate serotype-6 viral vectors. Conditioned media and cells were collected and homogenized prior to clarification through a 0.22 μm filter. Empty and full capsid particles were separated using CsCl centrifugation prior to re-suspension in a PBS solution containing 0.001% Poloxamer 188. Purified vector was then titered using a customized sequence-specific qPCR assay.

Animal Experiments—All animal experiments were performed by Myologica LLC at the University of Maryland Baltimore Veterinary Resources. Experiments were conducted in accordance to animal use protocols preapproved by the university's institutional animal care and use committee and according to National Institutes of Health guidelines. Wild-type C57BL/6 and C57BL/10ScSnJ as well as dystrophic C57BL/10ScSn-Dmdmdx/J (a.k.a. mdx) mice were obtained from Jackson Laboratory (Bar Harbor, MN). Animals were randomly assigned to treatment groups and technicians performing the studies were blinded to the treatments.

For local vector delivery, mice were anesthetized with isoflurane before making a single IM injection of 1×109 or 1×1010 vg. Vectors were diluted in Hank's buffered saline solution (HBSS) and directly injected into the tibialis anterior (TA) muscles. Needles were inserted in the caudal end of the muscle, just above the tendon moving up to the cranial. Injections began as the needle was slowly withdrawn, pausing occasionally to allow the solution (20 ml/muscle) to be absorbed. Control-injections of 20 ml carrier were also performed on the contralateral TA of each mouse. For systemic delivery studies, 5×1013 vg/kg in a 10 μl volume of HBSS was administered RO in 1 week-old neonates. Control mice were similarly injected with HBSS only. In some studies, muscle function was assessed after 8 or 12 weeks, as described below. All mice were eventually killed via thoracotomy under deep isoflurane anesthesia before excising, weighing and processing muscles.

Histology—Harvested muscles were placed in OCT cryoprotectant, frozen rapidly in dry ice-cooled isopentane and subsequently cryosectioned at 10 μm thickness starting at the mid-belly. Sections were fixed in 4% paraformaldehyde then stained with Alexa 647-labeled wheat germ agglutinin (Invitrogen/ThermoFisher Scientific, Waltham, MA) to identify sarcolemmal boundaries then mounted using ProLong Diamond Antifade Mountant with DAPI (Invitrogen/Fisher Scientific). Sections were then imaged using a Nikon (Melville, NY) Eclipse Ti2 microscope equipped with a Nikon DS-Qi2 monochrome camera and a Lumencor SpectraX light engine. Cell boundaries were all automatically traced with predictive software (Nikon Elements v4.51). These images were then used to determine the minimal Feret's diameter of each fiber and to quantify fibers with central nuclei using ImageJ software (National Institutes of Health, Bethesda, MD). All fibers of entire sections were assessed to avoid regional and user bias. To assess muscle fibrosis, sections were stained with a Masson's trichrome stain kit (Sigma-Aldrich, St. Louis, MO) according to manufacturer's instructions. Fibrosis was similarly quantified using ImageJ by normalizing blue-stained connective tissue area to the total area imaged.

Western blotting—Tissues were homogenized in T-Per (ThermoFisher Scientific, Waltham, MA) tissue protein extraction reagent supplemented with EDTA-free Pierce Protease and Phosphatase mini-tablets (ThermoFisher Scientific, Waltham, MA) in a NextAdvance Bullet Blender at maximum speed with 0.5 mm zirconium oxide beads. Samples were clarified by centrifugation at 13,000 xg for 10 minutes at 4° C. and protein concentrations were determined using a Pierce protein assay kit (ThermoFisher Scientific, Waltham, MA). Protein fractions (40 mg/lane) were separated by SDS-PAGE using pre-cast 4-20% tris-glycine gels (Bio-Rad, Hercules, CA) and electrotransferred onto Immobilon-FL PVDF membranes (MilliporeSigma, Burlington, MA) that were subsequently blocked in Intercept buffer (LI-COR, Lincoln, NE) before incubating with primary antibodies (Abcam, Cambridge, UK) for MADH7/Smad7 (1:500, ab226872) and GAPDH (1:10K, ab8245). Membranes were then washed in TBST before probing with goat-anti-rabbit 800CW and goat-anti-mouse 680RD secondary antibodies (LI-COR). Positive immunodetections were obtained and quantified using an Odyssey DLx immager (LI-COR). When appropriate, Smad7 protein levels were normalized to those of GAPDH, both as optical density units.

Assessment of muscle function-Muscle force/torque was assessed in vivo using a footplate assay and a 3050 muscle lever system (Aurora Scientific, Aurora, CAN) as described (Long et al., Hum Mol Genet 28, 1076-1089, 2019). Mice were anesthetized via inhalation (˜3% isoflurane, SomnoSuite, Kent Scientific) and placed on a thermostatically controlled table with anesthesia maintained via nose-cone (˜2% isoflurane). The knee was first isolated using a pin through the tibial head and the foot firmly fixed to a footplate on the motor shaft. For assessment of plantarflexor function, contractions were elicited by percutaneous electrical stimulation of the tibial nerve whereas the peroneal nerve was stimulated when assessing dorsiflexor function. A series of percutaneous electrical stimuli was initially used to establish the optimal isometric twitch torque. To avoid fatigue, muscles were stimulated with increasing current after a minimum rest interval of 30 s between each stimulus. Each muscle was then stimulated with a 0.2 ms pulse, 500 ms train duration at 1, 20, 50, 80, 100, and 150 Hz. Data were then analyzed using the manufacturer's software.

Statistical Analysis—Statistical differences were assessed across multiple conditions using one-way, two-way or repeated measures ANOVA tests with Tukey's post hoc test for multiple comparisons between group means. Differences between groups were reported as statistically significant for values of p<0.05. Data are presented as the mean±SEM. Correlation analyses were also performed, generating Pearson correlation coefficients (r) and two-tailed probability levels (p) for each relationship described.

### Example 2: Codon-Optimization of hSmad7 cDNA Removes Deleterious Structures Known to Impair Translation

According to particular aspects, codon-optimization of the hSmad7 cDNA sequence significantly improved the overall codon adaptation index (CAI) without altering a GC content that was near the maximum allowable (TABLE 1). TABLE 1 shows, according to particular aspects, the comparative codon analysis summary of ideal, wild-type and optimized hSmad7 cDNA sequences. Three separate algorithms were used to generate optimized sequences for human skeletal muscle. A multiple sequence alignment was then performed to generate a consensus sequence that was compared to the wild-type sequence in silico. Both sequences were then compared to the ideal characteristics listed. Codon-optimization improved the overall codon adaptation index and removed negative motifs and elements without significantly altering the overall GC %.

TABLE 1

| | | | | Antiviral | Neg cis | |
|---|---|---|---|---|---|---|
| Sequence | CAI | GC % | CFD % | motifs | elements | STOP |
| Ideal | 0.8-1 | 30-70 | <30 | 0 | 0 | TAA |
| WT | 0.78 | 64 | 4 | 3 | 3 | TAG |
| Optimized | 0.92 | 61.7 | 0 | 0 | 0 | TAA |

WT, wild-type; CAI, codon adaptation index: 0.8 is sufficient, 1.0 is perfect; GC, guanine/cytosine; CFD, codon frequency distribution of rare tandems; Neg cis, direct or inverted elements with the potential to cause secondary structure; STOP, translation-terminating stop codon

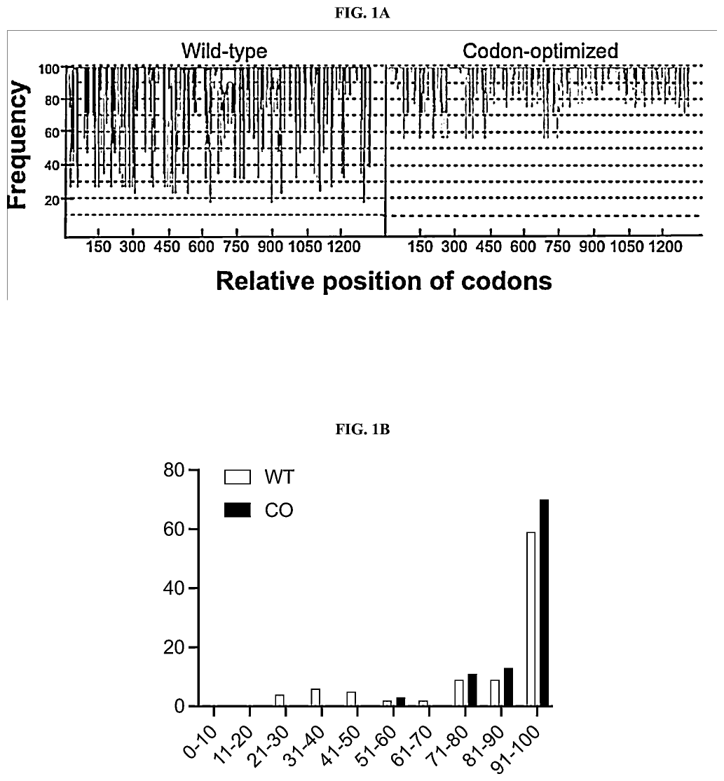

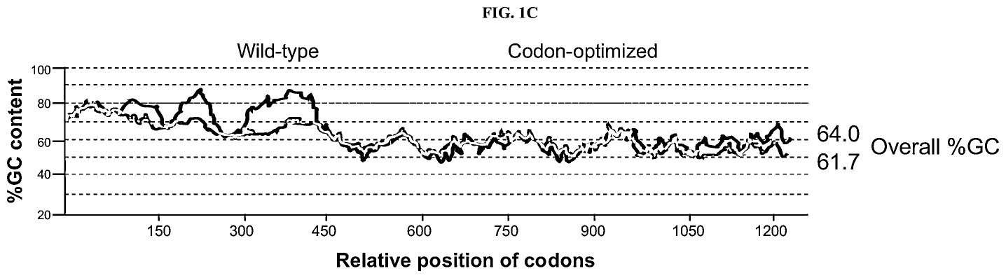

Changes also eliminated additional elements potentially causing secondary structure problems that reduce translation efficiency including a tandem codon repeat in the 3′-terminal region. The relatively conservative CAI change overall was still significant as several regions of concern were in fact substantially improved as illustrated with side-by-side comparisons of codon frequency relative to position (FIG. 1A). The relative percent of high-quality codons was also increased while that of low-quality codons was reduced (FIG. 1B). Three regions of exceptionally high GC content within the 5′-terminal region were adjusted to avoid secondary structure issues that could potentially interfere with ribosomal progression (FIG. 1C). Such changes were unique in intentionally maintaining overall GC content, which remained high but below the upper limit, while a comparison of the optimized and human Smad7 variant 1 cDNA sequences revealed 79% identity (FIG. 2). This particular variant is the primary Smad7 transcript expressed.

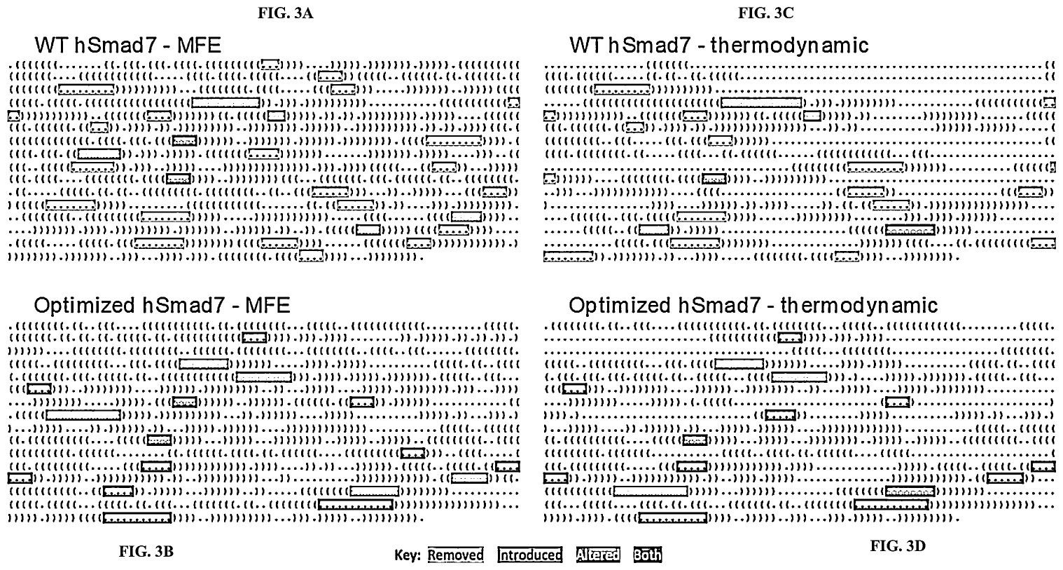

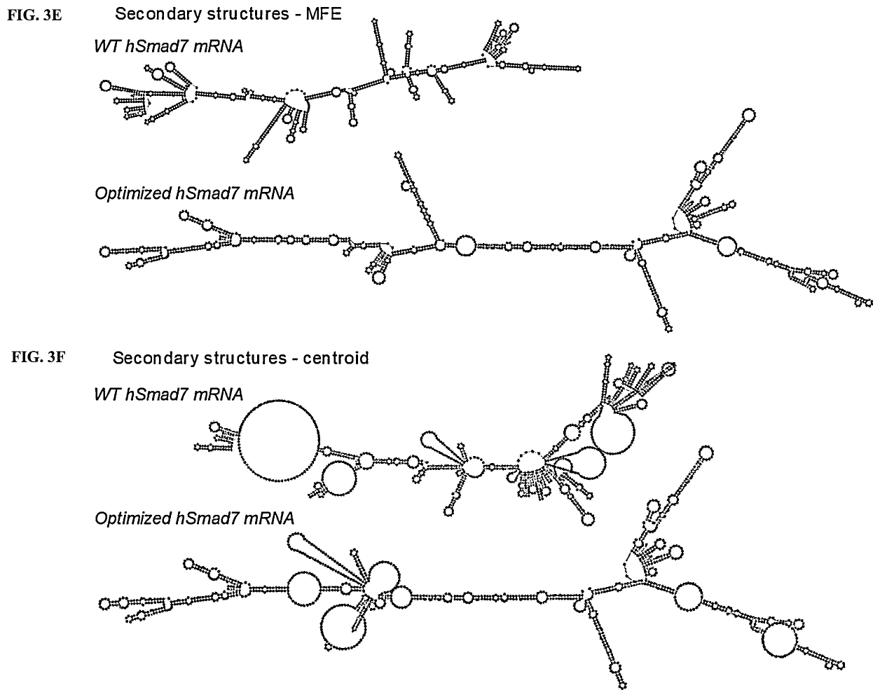

According to particular aspects, codon-optimization of the hSmad7 cDNA sequence significantly improved the overall secondary structure of hSmad7 mRNA by removing multiple hairpin motifs that can compromise ribosomal progression (FIGS. 3A-3F). Modeling at the minimal free energy (MFE) state identified 28 and 17 hairpins in the wild-type and codon-optimized sequences, respectively, for a net reduction of 11 hairpins with optimization (FIGS. 3A-3B). Thermodynamic modeling above MFE identified fewer hairpins as expected with a net reduction of 6 with optimization (FIGS. 3C-3D). Both models also computed secondary structures and revealed highly compact structures for wild-type hSmad7 with regions of low base-pairing probabilities (FIGS. 3E-3F). By contrast, the codon-optimized structures were less compact and contained mostly regions of high base-pairing probabilities. Such differences are reflected in diversity scores generated with thermodynamic modeling above MFE as the optimized score was 315.44 versus 439.34 for the wild-type sequence.

### Example 3: Comparability and Bioactivity of Codon-Optimized hSmad7 cDNA

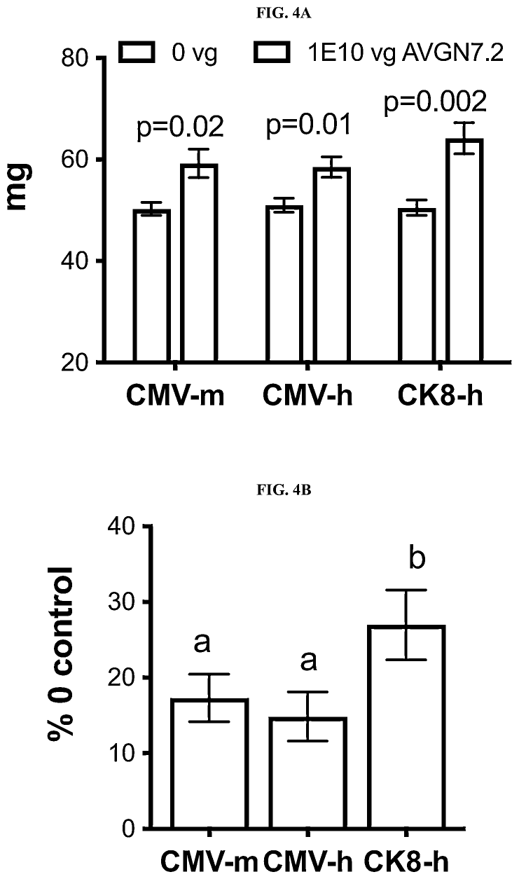

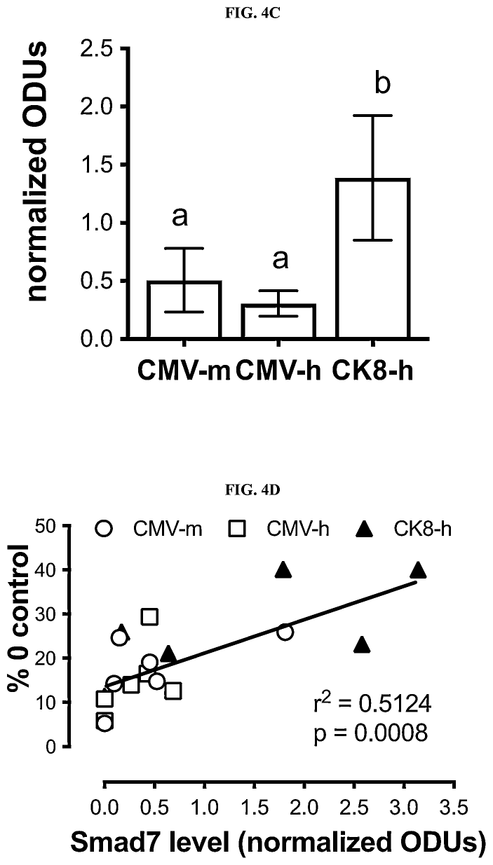

A contralateral control system that compared the efficacy of 3 vectors injected IM into the tibialis anterior (TA) muscles of mice. These vectors include rAAV6:CMV-mSmad7, the previously developed therapeutic containing a wild-type mouse Smad7 cDNA, and rAAV6:CMV-hSmad7 containing codon-optimized hSmad7 cDNA, both of which contained the CMV promoter. The third vector tested, rAAV6:CK8-hSmad7, also contained a codon-optimized hSmad7 cDNA as well as a promoter with high specificity and activity for striated muscle (Bengtsson et al., Nat Commun 8, 14454, 2017; Goncalves et al., Mol Ther 19, 1331-1341, 2011; Martari et al., Hum Gene Ther 20, 759-766, 2009). Although each increased muscle mass after just 4 weeks, statistical significance was greatest with rAAV6:CK8-hSmad7, which produced the largest difference and was therefore the most effective (FIGS. 4A-4B). Suboptimal doses were used to avoid maximizing efficacy and to help distinguish differences between vectors. Notwithstanding, Smad7 protein levels were higher in muscles injected with rAAV6:CK8-hSmad7 (FIG. 4C) likely due to the use of the muscle-specific CK8 promoter. In fact, hSmad7 protein levels were higher in TAs injected with rAAV6:CK8-hSmad7 than with either of the other two vectors while overall, the relative difference in mass between control and treated muscles were positively correlated to hSmad7 protein levels (FIG. 4D). These data together indicate that the combined use of a muscle-specific promoter and a codon-optimized cDNA sequence (i.e., rAAV6:CK8-hSmad7 ) is more effective than using the ubiquitous CMV promoter and a non-optimized sequence (i.e., rAAV6:CMV-mSmad7).

### Example 4: Codon-Optimized hSmad7 cDNA Increases Muscle Fiber Size in Healthy Wild-Type Mice

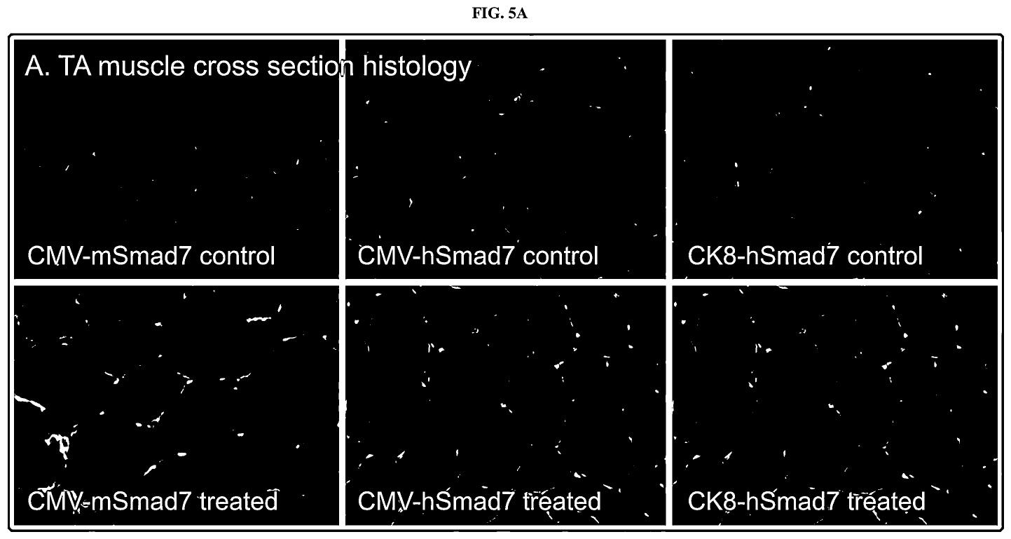

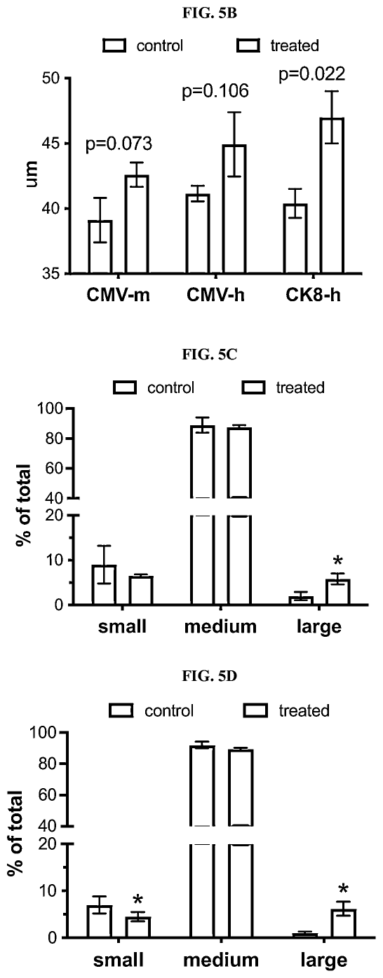

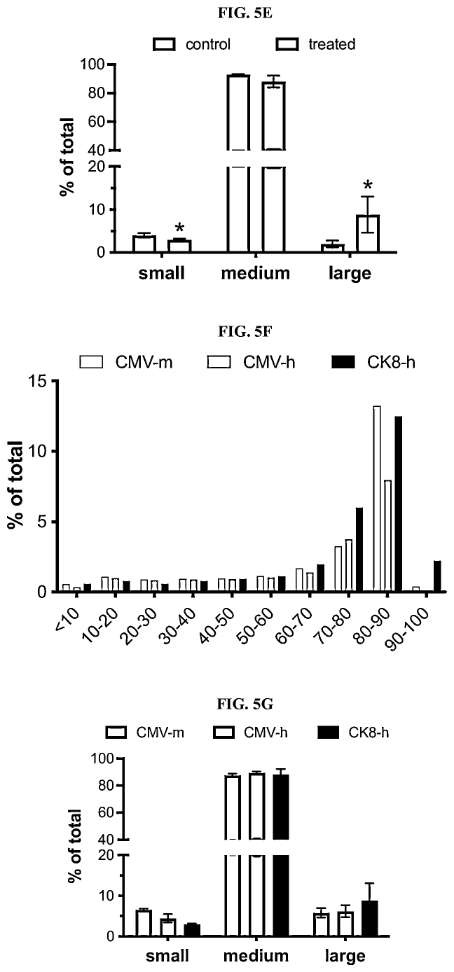

According to particular aspects, the differences in muscle mass (FIG. 4) were reflected in those of muscle fiber size as demonstrated qualitatively by sarcolemma and nuclear staining of muscle fibers (FIG. 5A). Differences were also noted when quantifying the size of all fibers (FIG. 5B), although a statistical difference was only noted with rAAV6:CK8-hSmad7. These results further illustrate the utility of using a suboptimal dose to distinguish differences between vectors. A more detailed analysis of fiber size distributions revealed a clear difference between control and treated muscles as well as between vectors. In fact, a shift in binned fiber sizes that includes fewer small fibers and/or more larger fibers was detected with each treatment group, although differences in both size groups were only detected in mice expressing the codon-optimized hSmad7 (FIG. 5C-E). Direct comparisons of fiber size distributions between vectors (FIG. 5F,G) failed to detect significant differences yet the trend of fewer small fibers and more larger fibers was again apparent in mice injected with rAAV6:CK8-hSmad7.

The histological, muscle mass and protein expression data all suggest that the combined use of CK8 and codon-optimized hSmad7 cDNA is superior. Using a human Smad7 sequence has the additional benefit of eliminating potential immune responses that could result from using the mouse ortholog in patients despite a high level of sequence similarity. Most importantly, these studies validate the use of codon-optimized hSmad7 by demonstrating equal or superior activity, as compared to that of rAAV6:CMV-mSmad7.

### Example 5: Codon-Optimized hSmad7 cDNA Enhances Muscle Function in Healthy Wild-Type Mice

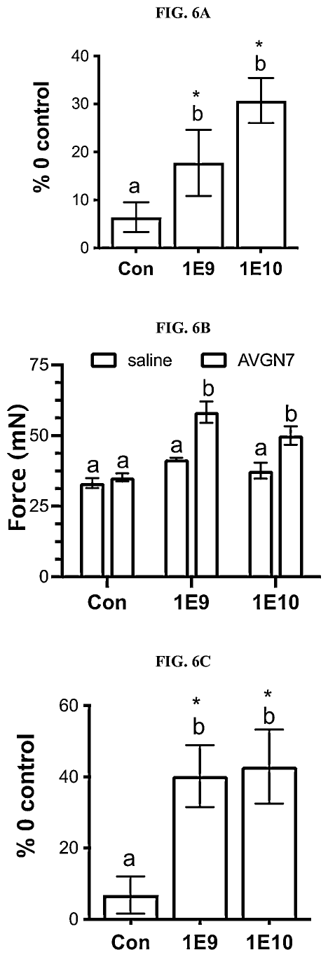

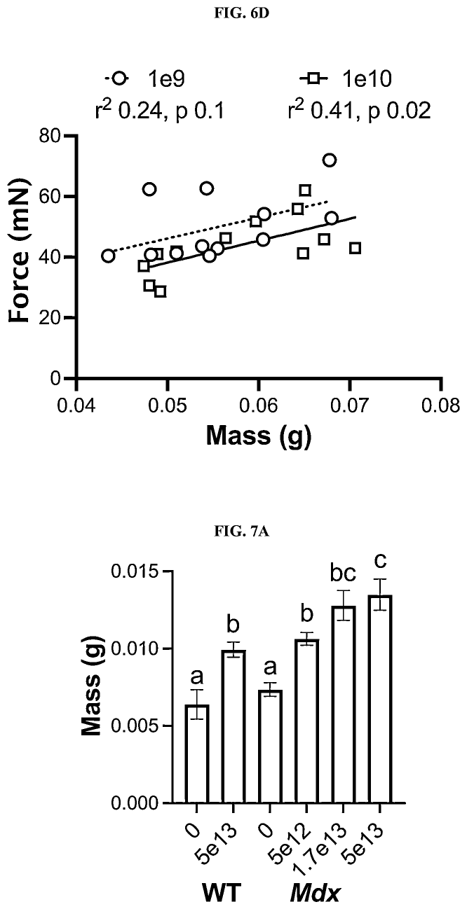

According to particular aspects, codon-optimized hSMAD7 enhanced the function of healthy muscle. Using a footplate assay that quantifies dorsiflexion force/torque and a contralateral control system, one TA muscle of each mouse was injected with saline and the other with one of two rAAV6:CK8-hSmad7 doses, 1×10⁹ or 1×10¹⁰ vg/muscle. A separate group of control mice received saline injections in both TA muscles. After 8 weeks, both doses of rAAV6:CK8-hSmad7 increased muscle mass significantly with the higher dose producing the greatest the effect (FIG. 6A). Both doses also increased dorsiflexion force over that of the contralateral control limbs and over both limbs of the control mice (FIG. 6B). The degree of change relative to the contralateral limbs was approximately 40% with both doses, suggesting that the minimal effective dose lies somewhere between (FIG. 6C). This is supported by similarly positive force-by-mass relationships for both groups and by the fact that the relationship was not statistically significant for the lower dose group (FIG. 6D). This combined ability to enhance muscle mass and function with local IM administration suggests that a gene therapy approach featuring codon-optimized hSmad7 could benefit patients with a peripheral neuropathy or nerve damage. Such insults induce muscle wasting locally (Cohen et al., Nat Rev Drug Discov 14, 58-74, 2015; Zhang et al., Med Hypotheses 69, 310-321, 2007) by activating muscle catabolic pathways suppressed by Smad7 overexpression (Winbanks et al., Science translational medicine 8, 348ra398, 2016), those that activate MuRF1 and MAFbx expression. Moreover, attenuating ActRII activation can improve muscle mass and function in animal models of these conditions.

### Example 6: Codon-Optimized hSmad7 cDNA Increases Muscle Mass and Fiber Size in Dystrophic Mice

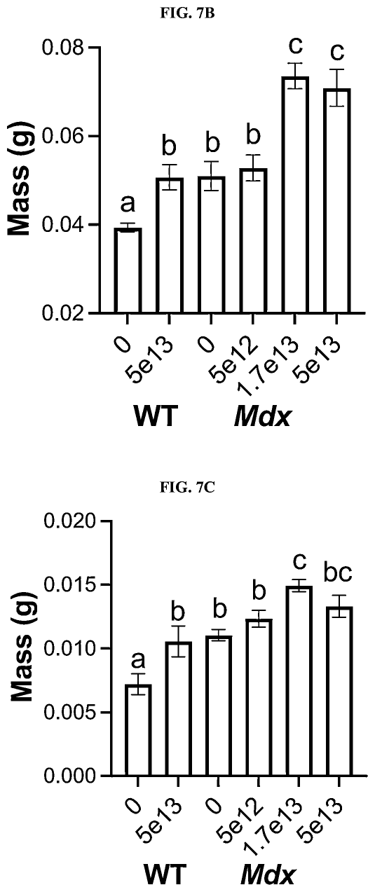

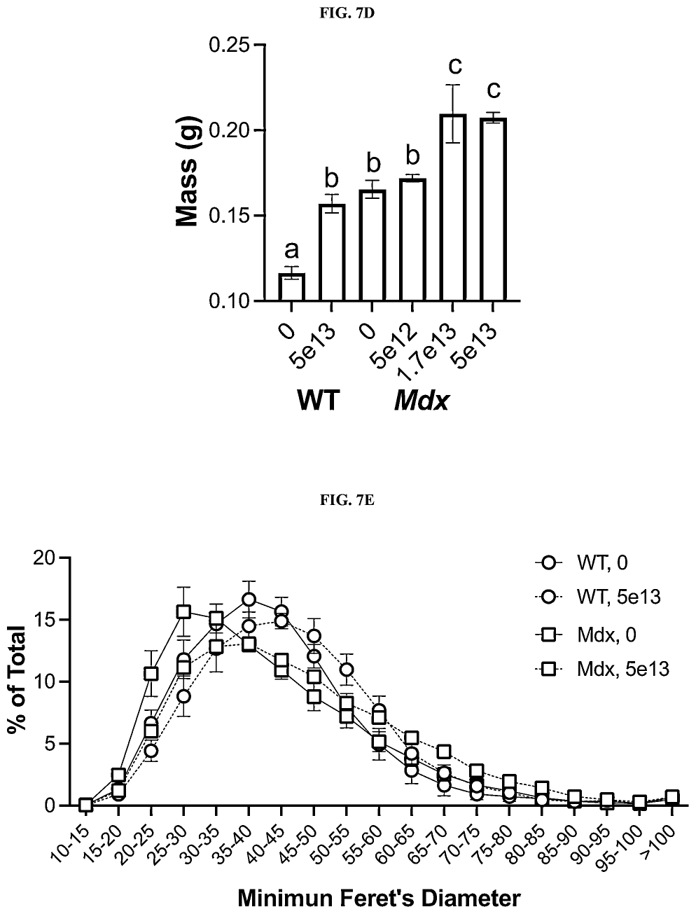

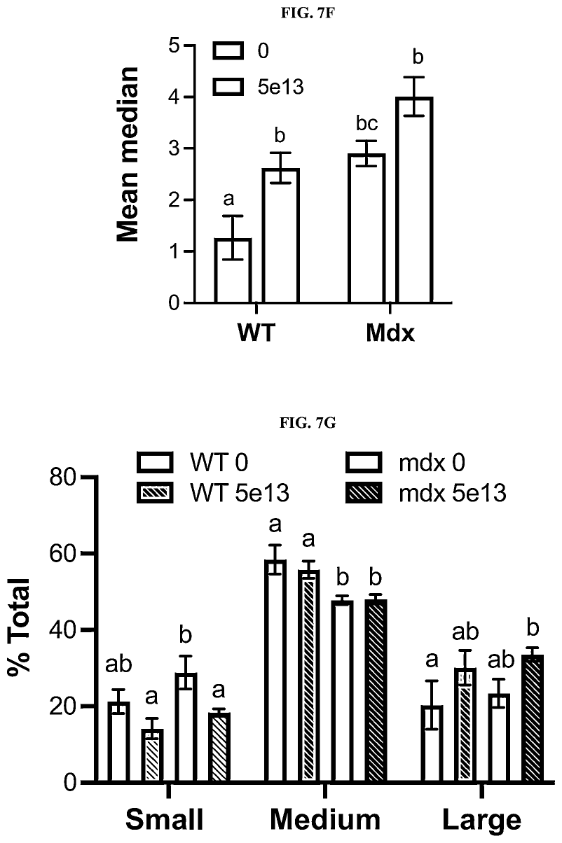

According to particular aspects, codon-optimized hSMAD7 increases the mass of dystrophic muscle (FIG. 7). Male mdx neonates, mouse models of Duchenne muscular dystrophy (DMD), were injected R.O. with 5×10¹³ vg/kg rAAV6:CK8-hSmad7 when 1 w.o. Mice were then evaluated after 12 weeks to mimic the young age that most DMD patients would likely be treated and evaluated. This systemic treatment increased the mass of different muscle groups independent of fiber/twitch type as predominantly type 1/slow (soleus), type 2B/fast (extensor digitorum longus & TA) and mixed fiber type (gastrocnemius) muscles were all significantly larger with treatment (FIGS. 7A-7D).

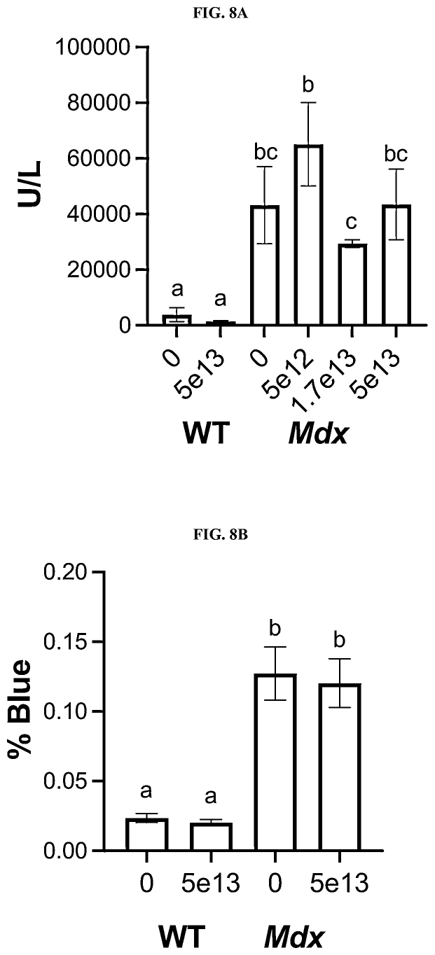

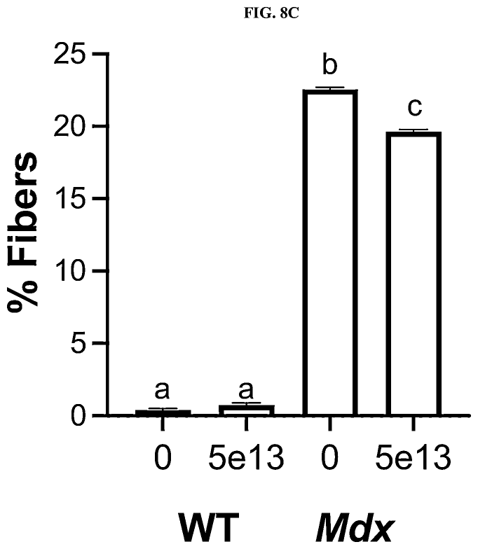

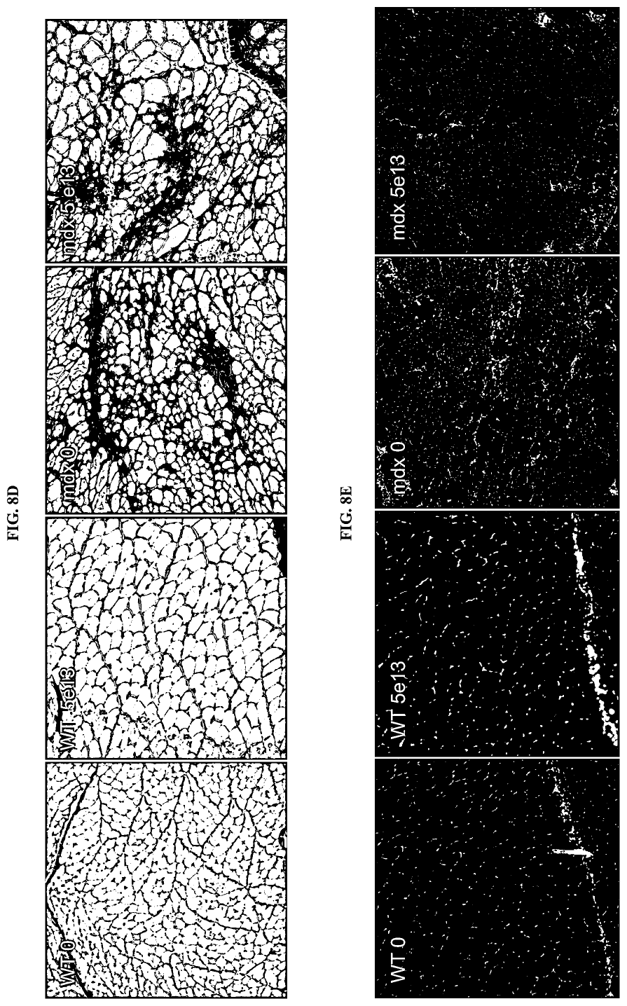

According to particular aspects, the changes in muscle mass with codon-optimized hSmad7overexpression were accompanied by a similar change in muscle fiber size (FIGS. 7E-7G). This was reflected in the fiber size distribution (FIG. 7E), in the average median fiber size (FIG. 7F) and in the fact that there were fewer small and more large fibers in treated muscles (FIG. 7G). Note that fiber size variability was higher in untreated mdx muscles compared to wild-type, consistent with the early onset hypertrophy that develops with DMD. Nevertheless, rAAV6:CK8-hSmad7 was still capable of increasing muscle mass and fiber size despite evidence of pre-existing disease-induced hypertrophy. Levels of serum creatine kinase and muscle fibrosis remained elevated in all mdx groups assessed, regardless of treatment (FIGS. 8A-8B). Central nucleation in mdx muscle, which results from increased muscle regeneration and satellite cell fusion with damaged fibers, was slightly yet significantly reduced with treatment (FIG. 8C). These differences were readily apparent histologically (FIGS. 8D-8E) and suggest that codon-optimized hSmad7 enhances muscle cell and fiber size without exacerbating the underlying dystrophic pathology.

### Example 7: Codon-Optimized hSmad7 cDNA Enhances Muscle Function in Dystrophic Mice

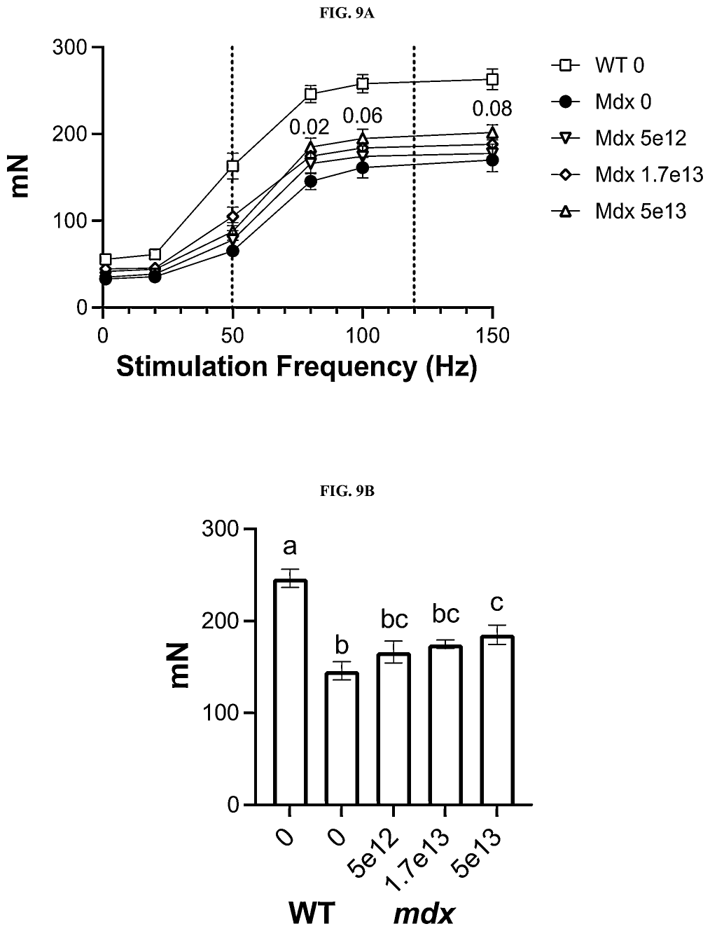

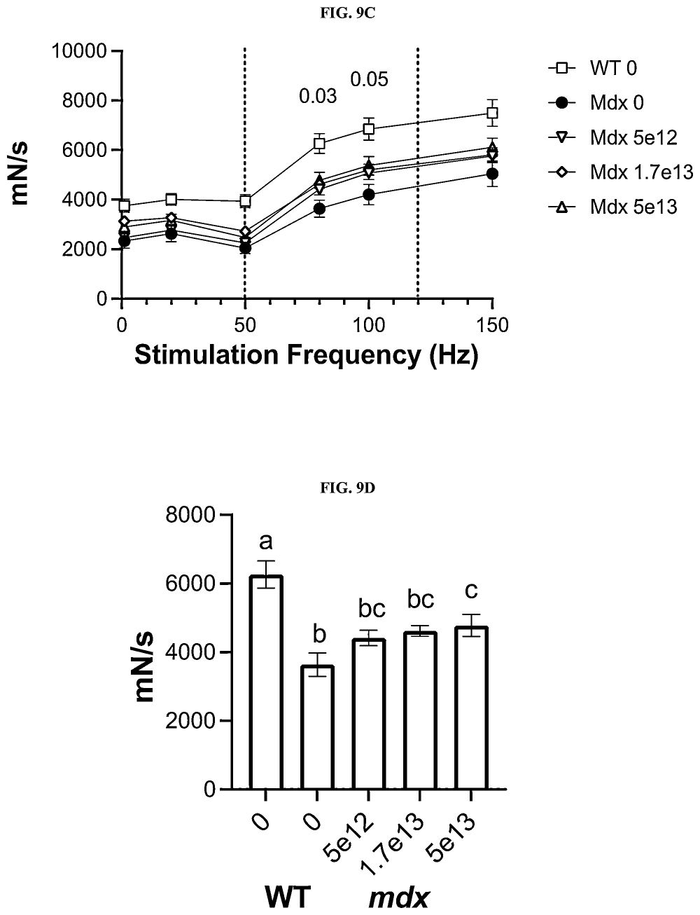

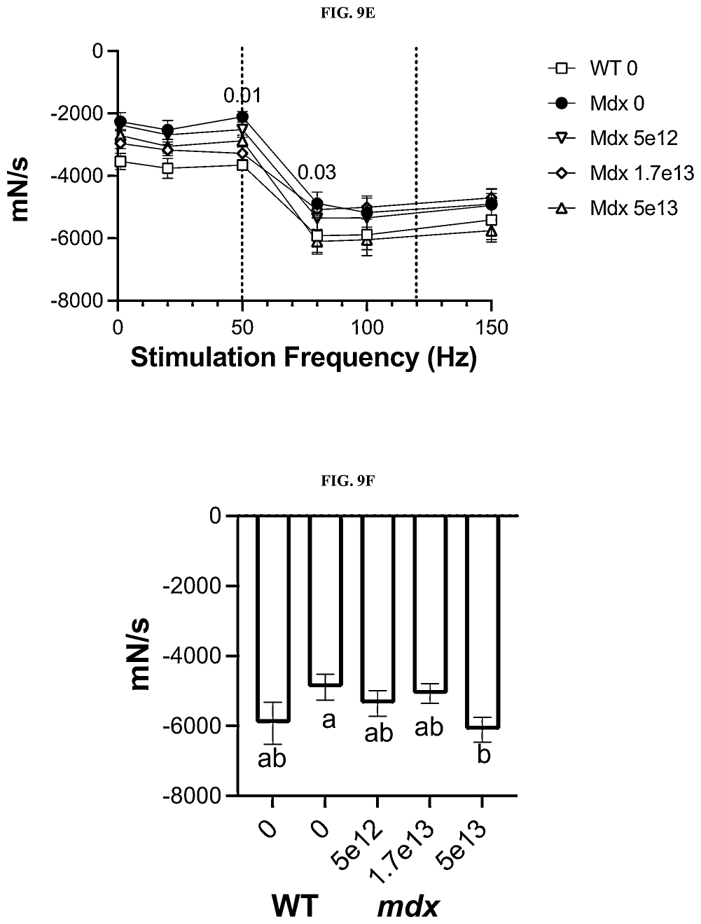

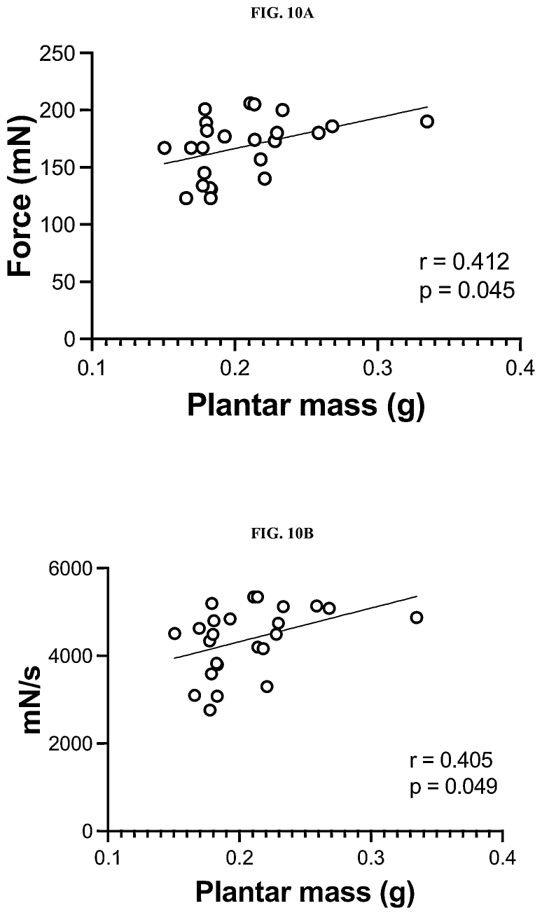

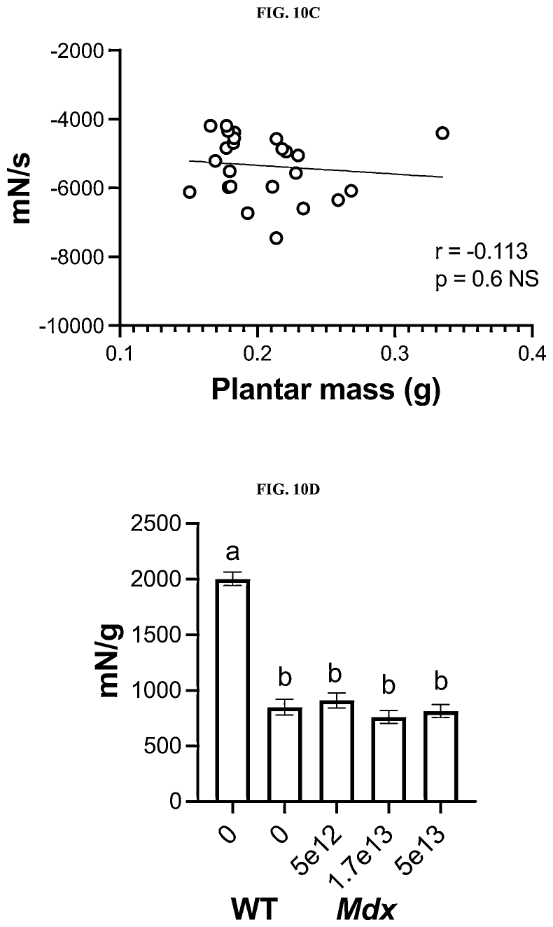

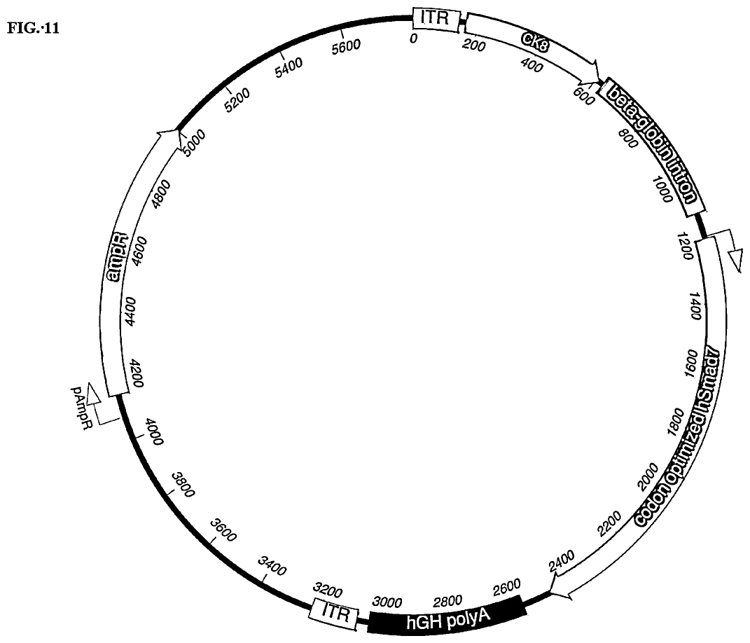

According to particular aspects, systemic administration of a codon-optimized hSMAD7 gene therapy increases the function of dystrophic muscle (FIGS. 9A-9F). Mdx and wild-type mice were treated with or without rAAV6:CK8-hSmad7 and plantarflexor force/torque was quantified using a footplate assay. Sciatic nerves were directly stimulated with increasing frequencies that spanned the normal in vivo physiological range of 50 to 120 Hz and were additionally stimulated at 150 Hz to assure that maximal or near-maximal activation was reached within the physiological range. Expressing codon-optimized hSmad7 increased total force within the physiological range and in a dose-dependent manner (FIG. 9A). Dose-dependency was best demonstrated at 80 Hz where the force generated by mdx mice treated with 1e14 vg/kg was greater than that generated by the mdx controls, but not greater than the forces from mice receiving lower doses (FIG. 9B). Similar patterns were also observed when quantifying contraction and relaxation rates, indicating that total force as well as the rate of force development were augmented by codon-optimized hSmad7 (FIGS. 9C-9F). Although mass/force relationships are often difficult to demonstrate and are discordant in dystrophic animals and patients where muscles become hypertrophied from enhanced muscle regeneration, fibrosis and inflammation, correlation analysis indicates positive mass/force and mass/contraction rate relationships with treatment in mdx mice (FIG. 10A-10B). Moreover, differences in total force at 80 Hz were lost after normalization to tissue mass (FIG. 10D). These data together indicate that the enhanced muscle force is a result of similarly enhanced muscle mass.

According to particular aspects, the enhanced muscle mass and function in mdx mice administered rAAV6:CK8-hSmad7 compares favorably with the anti-catabolic effects of targeting ActRII ligands in mdx mice (Pistilli et al., Am J Pathol 178, 1287-1297, 2011; Morine et al., Muscle Nerve 42, 722-730, 2010; Qiao et al., Hum Gene Ther 19, 241-254, 2008; Bogdanovich et al., Faseb J 19, 543-549, 2005; Bogdanovich et al., Nature 420, 418-421, 2002) and in models of other muscular dystrophies and myopathies (Bogdanovich et al., Muscle Nerve 37, 308-316, 2008; Pearsall et al., Sci Rep 9, 11392, 2019; Harish et al., Journal of cachexia, sarcopenia and muscle 10, 1016-1026, 2019). Targeting ActRII ligands, which include the activins (Act-A, Act-B & Act-A/B), myostatin, GDF11 and others, presents opportunities for serious off-target effects as these ligands regulate a variety of systems (e.g. reproduction, neurogenesis, angiogenesis, osteogenesis, etc.) and recognize multiple receptors in addition to ActRII (Rodgers and Ward Endocr Rev 2021). The ligand approach can produce effects outside of muscle. Indeed, a clinical trial of an ActRII ligand-trap administered to boys with DMD was terminated prematurely due to serious off-target effects that compromised respiratory epithelium (Campbell et al., Muscle Nerve 55, 458-464, 2017) while a monoclonal antibody recognizing the ActRII ligand binding domain was demonstrated to alter pituitary function (Garito et al., Clin Endocrinol (Oxf) 88, 908-919, 2018).

### Example 8: Enhancing Muscle Mass and/or Strength using Codon-Optimized Smad7

This example describes an exemplary method for the clinical use of rAAV vectors encoding codon-optimized hSmad7 for the treatment of muscle wasting.

A patient diagnosed with muscle wasting, as occurs with muscular dystrophies and myopathies, peripheral neuropathies, neuromuscular disease and many other chronic disease states, is selected for treatment. The patient is administered a therapeutically effective amount of a recombinant AAV expressing codon-optimized hSmad7, such as a rAAV comprising SEQ ID NO:1 or SEQ ID NO:2, as disclosed herein. The recombinant AAV can be administered intravenously or intramuscularly. An appropriate therapeutic dose can be selected by a medical practitioner. In some cases, the therapeutically effective dose is in the range of 1×10⁹ to 1×10¹⁴ vector genomes (vg)/kg, such as about 1×10¹¹ or 1×10¹² vg/kg. In most instances, the patient is administered a single dose. In the absence of immuno-modulation, the patient is likely to tolerate only a single infusion of rAAV. If the subject has had pre-exposure immunomodulation, two or more doses may be administered. The health of the subject can be monitored over time to determine the effectiveness of the treatment.

In view of the many possible embodiments to which the principles of the disclosed invention may be applied, it should be recognized that the illustrated embodiments are only preferred examples of the invention and should not be taken as limiting the scope of the invention. Rather, the scope of the invention is defined by the following claims. We therefore claim as our invention all that comes within the scope and spirit of these claims.

SEQ ID NO. 1: Nucleic acid sequence of codon-optimized human Smad7 cDNA
ATGTTTCGGACAAAGAGAAGCGCACTGGTCAGAAGACTGTGGAGAAGCAGAGCACCTGGAGGGGAGGA
TGAAGAAGAGGGAGCCGGGGGCGGCGGCGGCGGCGGCGAGCTGCGGGGCGAGGGCGCCACCGACAGCA
GAGCCCACGGCGCCGGCGGCGGCGGCCCCGGCAGGGCCGGCTGCTGTCTGGGCAAGGCCGTGCGCGGC
GCCAAGGGCCACCACCACCCTCACCCCCCTGCAGCAGGAGCAGGAGCAGCAGGAGGAGCAGAGGCCGA
TCTGAAGGCCCTGACACACTCTGTGCTGAAGAAGCTGAAGGAGAGGCAGCTGGAGCTGCTGCTGCAGG
CCGTGGAGAGCAGGGGCGGCACCCGCACAGCCTGTCTGCTGCTGCCCGGCCGGCTGGACTGCAGACTG
GGACCAGGAGCACCAGCAGGAGCACAGCCAGCCCAGCCACCCAGCTCCTATTCCCTGCCCCTGCTGCT
GTGCAAGGTGTTCAGGTGGCCTGATCTGCGCCACTCTAGCGAGGTGAAGCGGCTGTGCTGTTGCGAGA
GCTACGGCAAGATCAACCCAGAGCTGGTGTGCTGCAATCCCCACCACCTGTCCAGGCTGTGCGAGCTG
GAGAGCCCTCCACCACCTTACTCCCGCTATCCCATGGACTTTCTGAAGCCTACCGCAGACTGCCCTGA
TGCAGTGCCATCCTCTGCCGAGACAGGAGGAACAAACTATCTGGCACCTGGAGGCCTGTCTGACAGCC
AGCTGCTGCTGGAGCCAGGCGATAGGTCCCACTGGTGCGTGGTGGCCTACTGGGAGGAGAAGACACGG
GTGGGCAGACTGTATTGCGTGCAGGAGCCTTCTCTGGACATCTTCTACGATCTGCCACAGGGCAATGG
CTTTTGTCTGGGCCAGCTGAACTCCGACAATAAGTCTCAGCTGGTGCAGAAGGTGCGCAGCAAGATCG
GATGCGGAATCCAGCTGACCAGGGAGGTGGATGGCGTGTGGGTGTACAACAGAAGCTCCTATCCAATC
TTCATCAAGTCTGCCACCCTGGACAATCCCGATAGCCGGACACTGCTGGTGCACAAGGTGTTCCCCGG
CTTTTCCATCAAGGCCTTCGACTACGAGAAGGCCTATTCTCTGCAGCGGCCCAACGATCACGAGTTTA
TGCAGCAGCCTTGGACCGGCTTCACAGTGCAGATCTCCTTTGTGAAGGGCTGGGGCCAGTGCTACACT
CGGCAGTTCATTTCATCCTGTCCTTGTTGGCTGGAAGTGATTTTCAATTCTCGGTAA
SEQ ID NO. 2: Nucleic acid sequence of pAAV-CK8-hSmad7.opt
CCTGCAGGCAGCTGCGCGCTCGCTCGCTCACTGAGGCCGCCCGGGCAAAGCCCGGGCGTCGGGCGACC
TTTGGTCGCCCGGCCTCAGTGAGCGAGCGAGCGCGCAGAGAGGGAGTGGCCAACTCCATCACTAGGGG
TTCCTGCGGCCGCACGCGTACGCTAGCTAGACTAGCATGCTGCCCATGTAAGGAGGCAAGGCCTGGGG
ACACCCGAGATGCCTGGTTATAATTAACCCAGACATGTGGCTGCCCCCCCCCCCCCAACACCTGCTGC
CTCTAAAAATAACCCTGCATGCCATGTTCCCGGCGAAGGGCCAGCTGTCCCCCGCCAGCTAGACTCAG
CACTTAGTTTAGGAACCAGTGAGCAAGTCAGCCCTTGGGGCAGCCCATACAAGGCCATGGGGCTGGGC
AAGCTGCACGCCTGGGTCCGGGGTGGGCACGGTGCCCGGGCAACGAGCTGAAAGCTCATCTGCTCTCA
GGGGCCCCTCCCTGGGGACAGCCCCTCCTGGCTAGTCACACCCTGTAGGCTCCTCTATATAACCCAGG
GGCACAGGGGCTGCCCTCATTCTACCACCACCTCCACAGCACAGACAGACACTCAGGAGCCAGCCAGC
GTATACGCGGATTCGAATCCCGGCCGGGAACGGTGCATTGGAACGCGGATTCCCCGTGCCAAGAGTGA
CGTAAGTACCGCCTATAGAGTCTATAGGCCCACAAAAAATGCTTTCTTCTTTTAATATACTTTTTTGT
TTATCTTATTTCTAATACTTTCCCTAATCTCTTTCTTTCAGGGCAATAATGATACAATGTATCATGCC
TCTTTGCACCATTCTAAAGAATAACAGTGATAATTTCTGGGTTAAGGCAATAGCAATATTTCTGCATA
TAAATATTTCTGCATATAAATTGTAACTGATGTAAGAGGTTTCATATTGCTAATAGCAGCTACAATCC
AGCTACCATTCTGCTTTTATTTTATGGTTGGGATAAGGCTGGATTATTCTGAGTCCAAGCTAGGCCCT
TTTGCTAATCATGTTCATACCTCTTATCTTCCTCCCACAGCTCCTGGGCAACGTGCTGGTCTGTGTGC
TGGCCCATCACTTTGGCAAAGAATTGGGATTCGAACATCGATTGAATTCGCCCTTGTCATGTTCGCTC
CTTAGCCGGCAAACGACTTTTCTCCTCGCCTCCTCGGCCACCATGTTTCGGACAAAGAGAAGCGCACT
GGTCAGAAGACTGTGGAGAAGCAGAGCACCTGGAGGGGAGGATGAAGAAGAGGGAGCCGGGGGGGGCG
GCGGCGGCGGCGAGCTGCGGGGCGAGGGCGCCACCGACAGCAGAGCCCACGGCGCCGGCGGCGGCGGC
CCCGGCAGGGCCGGCTGCTGTCTGGGCAAGGCCGTGCGCGGCGCCAAGGGCCACCACCACCCTCACCC
CCCTGCAGCAGGAGCAGGAGCAGCAGGAGGAGCAGAGGCCGATCTGAAGGCCCTGACACACTCTGTGC
TGAAGAAGCTGAAGGAGAGGCAGCTGGAGCTGCTGCTGCAGGCCGTGGAGAGCAGGGGCGGCACCCGC
ACAGCCTGTCTGCTGCTGCCCGGCCGGCTGGACTGCAGACTGGGACCAGGAGCACCAGCAGGAGCACA
GCCAGCCCAGCCACCCAGCTCCTATTCCCTGCCCCTGCTGCTGTGCAAGGTGTTCAGGTGGCCTGATC
TGCGCCACTCTAGCGAGGTGAAGCGGCTGTGCTGTTGCGAGAGCTACGGCAAGATCAACCCAGAGCTG
GTGTGCTGCAATCCCCACCACCTGTCCAGGCTGTGCGAGCTGGAGAGCCCTCCACCACCTTACTCCCG
CTATCCCATGGACTTTCTGAAGCCTACCGCAGACTGCCCTGATGCAGTGCCATCCTCTGCCGAGACAG
GAGGAACAAACTATCTGGCACCTGGAGGCCTGTCTGACAGCCAGCTGCTGCTGGAGCCAGGCGATAGG
TCCCACTGGTGCGTGGTGGCCTACTGGGAGGAGAAGACACGGGTGGGCAGACTGTATTGCGTGCAGGA
GCCTTCTCTGGACATCTTCTACGATCTGCCACAGGGCAATGGCTTTTGTCTGGGCCAGCTGAACTCCG
ACAATAAGTCTCAGCTGGTGCAGAAGGTGCGCAGCAAGATCGGATGCGGAATCCAGCTGACCAGGGAG
GTGGATGGCGTGTGGGTGTACAACAGAAGCTCCTATCCAATCTTCATCAAGTCTGCCACCCTGGACAA
TCCCGATAGCCGGACACTGCTGGTGCACAAGGTGTTCCCCGGCTTTTCCATCAAGGCCTTCGACTACG
AGAAGGCCTATTCTCTGCAGCGGCCCAACGATCACGAGTTTATGCAGCAGCCTTGGACCGGCTTCACA
GTGCAGATCTCCTTTGTGAAGGGCTGGGGCCAGTGCTACACTCGGCAGTTCATTTCATCCTGTCCTTG
TTGGCTGGAAGTGATTTTCAATTCTCGGTAATCGGTCGTGTGGTGAAGGGCGAATTCCCCGGGGATCC
TCTAGAGTCGAGCTGCAGAAGCTTGCCTCGAGCAGCGCTGCTCGAGAGATCTACGGGTGGCATCCCTG
TGACCCCTCCCCAGTGCCTCTCCTGGCCCTGGAAGTTGCCACTCCAGTGCCCACCAGCCTTGTCCTAA
TAAAATTAAGTTGCATCATTTTGTCTGACTAGGTGTCCTTCTATAATATTATGGGGTGGAGGGGGGTG
GTATGGAGCAAGGGGCAAGTTGGGAAGACAACCTGTAGGGCCTGCGGGGTCTATTGGGAACCAAGCTG
GAGTGCAGTGGCACAATCTTGGCTCACTGCAATCTCCGCCTCCTGGGTTCAAGCGATTCTCCTGCCTC
AGCCTCCCGAGTTGTTGGGATTCCAGGCATGCATGACCAGGCTCAGCTAATTTTTGTTTTTTTGGTAG
AGACGGGGTTTCACCATATTGGCCAGGCTGGTCTCCAACTCCTAATCTCAGGTGATCTACCCACCTTG
GCCTCCCAAATTGCTGGGATTACAGGCGTGAACCACTGCTCCCTTCCCTGTCCTTCTGATTTTGTAGG
TAACCACGTGCGGACCGAGCGGCCGCAGGAACCCCTAGTGATGGAGTTGGCCACTCCCTCTCTGCGCG
CTCGCTCGCTCACTGAGGCCGGGCGACCAAAGGTCGCCCGACGCCCGGGCTTTGCCCGGGCGGCCTCA
GTGAGCGAGCGAGCGCGCAGCTGCCTGCAGGGGCGCCTGATGCGGTATTTTCTCCTTACGCATCTGTG
CGGTATTTCACACCGCATACGTCAAAGCAACCATAGTACGCGCCCTGTAGCGGCGCATTAAGCGCGGC
GGGTGTGGTGGTTACGCGCAGCGTGACCGCTACACTTGCCAGCGCCCTAGCGCCCGCTCCTTTCGCTT
TCTTCCCTTCCTTTCTCGCCACGTTCGCCGGCTTTCCCCGTCAAGCTCTAAATCGGGGGCTCCCTTTA
GGGTTCCGATTTAGTGCTTTACGGCACCTCGACCCCAAAAAACTTGATTTGGGTGATGGTTCACGTAG
TGGGCCATCGCCCTGATAGACGGTTTTTCGCCCTTTGACGTTGGAGTCCACGTTCTTTAATAGTGGAC
TCTTGTTCCAAACTGGAACAACACTCAACCCTATCTCGGGCTATTCTTTTGATTTATAAGGGATTTTG
CCGATTTCGGCCTATTGGTTAAAAAATGAGCTGATTTAACAAAAATTTAACGCGAATTTTAACAAAAT
ATTAACGTTTACAATTTTATGGTGCACTCTCAGTACAATCTGCTCTGATGCCGCATAGTTAAGCCAGC
CCCGACACCCGCCAACACCCGCTGACGCGCCCTGACGGGCTTGTCTGCTCCCGGCATCCGCTTACAGA
CAAGCTGTGACCGTCTCCGGGAGCTGCATGTGTCAGAGGTTTTCACCGTCATCACCGAAACGCGCGAG
ACGAAAGGGCCTCGTGATACGCCTATTTTTATAGGTTAATGTCATGATAATAATGGTTTCTTAGACGT
CAGGTGGCACTTTTCGGGGAAATGTGCGCGGAACCCCTATTTGTTTATTTTTCTAAATACATTCAAAT
ATGTATCCGCTCATGAGACAATAACCCTGATAAATGCTTCAATAATATTGAAAAAGGAAGAGTATGAG
TATTCAACATTTCCGTGTCGCCCTTATTCCCTTTTTTGCGGCATTTTGCCTTCCTGTTTTTGCTCACC
CAGAAACGCTGGTGAAAGTAAAAGATGCTGAAGATCAGTTGGGTGCACGAGTGGGTTACATCGAACTG
GATCTCAACAGCGGTAAGATCCTTGAGAGTTTTCGCCCCGAAGAACGTTTTCCAATGATGAGCACTTT
TAAAGTTCTGCTATGTGGCGCGGTATTATCCCGTATTGACGCCGGGCAAGAGCAACTCGGTCGCCGCA
TACACTATTCTCAGAATGACTTGGTTGAGTACTCACCAGTCACAGAAAAGCATCTTACGGATGGCATG
ACAGTAAGAGAATTATGCAGTGCTGCCATAACCATGAGTGATAACACTGCGGCCAACTTACTTCTGAC
AACGATCGGAGGACCGAAGGAGCTAACCGCTTTTTTGCACAACATGGGGGATCATGTAACTCGCCTTG
ATCGTTGGGAACCGGAGCTGAATGAAGCCATACCAAACGACGAGCGTGACACCACGATGCCTGTAGCA
ATGGCAACAACGTTGCGCAAACTATTAACTGGCGAACTACTTACTCTAGCTTCCCGGCAACAATTAAT
AGACTGGATGGAGGCGGATAAAGTTGCAGGACCACTTCTGCGCTCGGCCCTTCCGGCTGGCTGGTTTA
TTGCTGATAAATCTGGAGCCGGTGAGCGTGGGTCTCGCGGTATCATTGCAGCACTGGGGCCAGATGGT
AAGCCCTCCCGTATCGTAGTTATCTACACGACGGGGAGTCAGGCAACTATGGATGAACGAAATAGACA
GATCGCTGAGATAGGTGCCTCACTGATTAAGCATTGGTAACTGTCAGACCAAGTTTACTCATATATAC
TTTAGATTGATTTAAAACTTCATTTTTAATTTAAAAGGATCTAGGTGAAGATCCTTTTTGATAATCTC
ATGACCAAAATCCCTTAACGTGAGTTTTCGTTCCACTGAGCGTCAGACCCCGTAGAAAAGATCAAAGG
ATCTTCTTGAGATCCTTTTTTTCTGCGCGTAATCTGCTGCTTGCAAACAAAAAAACCACCGCTACCAG
CGGTGGTTTGTTTGCCGGATCAAGAGCTACCAACTCTTTTTCCGAAGGTAACTGGCTTCAGCAGAGCG
CAGATACCAAATACTGTACTTCTAGTGTAGCCGTAGTTAGGCCACCACTTCAAGAACTCTGTAGCACC
GCCTACATACCTCGCTCTGCTAATCCTGTTACCAGTGGCTGCTGCCAGTGGCGATAAGTCGTGTCTTA
CCGGGTTGGACTCAAGACGATAGTTACCGGATAAGGCGCAGCGGTCGGGCTGAACGGGGGGTTCGTGC
ACACAGCCCAGCTTGGAGCGAACGACCTACACCGAACTGAGATACCTACAGCGTGAGCTATGAGAAAG
CGCCACGCTTCCCGAAGGGAGAAAGGCGGACAGGTATCCGGTAAGCGGCAGGGTCGGAACAGGAGAGC
GCACGAGGGAGCTTCCAGGGGGAAACGCCTGGTATCTTTATAGTCCTGTCGGGTTTCGCCACCTCTGA
CTTGAGCGTCGATTTTTGTGATGCTCGTCAGGGGGGCGGAGCCTATGGAAAAACGCCAGCAACGCGGC
CTTTTTACGGTTCCTGGCCTTTTGCTGGCCTTTTGCTCACATGT
SEQ ID NO. 3: Amino acid sequence of human Smad7 protein
MFRTKRSALVRRLWRSRAPGGEDEEEGAGGGGGGGELRGEGATDSRAHGAGGGGPGRAGCCLGKAVRG
AKGHHHPHPPAAGAGAAGGAEADLKALTHSVLKKLKERQLELLLQAVESRGGTRTACLLLPGRLDCRL
GPGAPAGAQPAQPPSSYSLPLLLCKVFRWPDLRHSSEVKRLCCCESYGKINPELVCCNPHHLSRLCEL
ESPPPPYSRYPMDFLKPTADCPDAVPSSAETGGTNYLAPGGLSDSQLLLEPGDRSHWCVVAYWEEKTR
VGRLYCVQEPSLDIFYDLPQGNGFCLGQLNSDNKSQLVQKVRSKIGCGIQLTREVDGVWVYNRSSYPI
FIKSATLDNPDSRTLLVHKVFPGFSIKAFDYEKAYSLQRPNDHEFMQQPWTGFTVQISFVKGWGQCYT
RQFISSCPCWLEVIFNSR
SEQ ID NO. 4: Nucleic acid sequence of wildtype human Smad7 cDNA variant 1
ATGTTCAGGACCAAACGATCTGCGCTCGTCCGGCGTCTCTGGAGGAGCCGTGCGCCCGGCGGCGAGGA
CGAGGAGGAGGGCGCAGGGGGAGGTGGAGGAGGAGGCGAGCTGCGGGGAGAAGGGGCGACGGACAGCC
GAGCGCATGGGGCCGGTGGCGGCGGCCCGGGCAGGGCTGGATGCTGCCTGGGCAAGGCGGTGCGAGGT
GCCAAAGGTCACCACCATCCCCACCCGCCAGCCGCGGGCGCCGGCGCGGCCGGGGGCGCCGAGGCGGA
TCTGAAGGCGCTCACGCACTCGGTGCTCAAGAAACTGAAGGAGCGGCAGCTGGAGCTGCTGCTCCAGG
CCGTGGAGTCCCGCGGCGGGACGCGCACCGCGTGCCTCCTGCTGCCCGGCCGCCTGGACTGCAGGCTG
GGCCCGGGGGCGCCCGCCGGCGCGCAGCCTGCGCAGCCGCCCTCGTCCTACTCGCTCCCCCTCCTGCT
GTGCAAAGTGTTCAGGTGGCCGGATCTCAGGCATTCCTCGGAAGTCAAGAGGCTGTGTTGCTGTGAAT
CTTACGGGAAGATCAACCCCGAGCTGGTGTGCTGCAACCCCCATCACCTTAGCCGACTCTGCGAACTA
GAGTCTCCCCCCCCTCCTTACTCCAGATACCCGATGGATTTTCTCAAACCAACTGCAGACTGTCCAGA
TGCTGTGCCTTCCTCCGCTGAAACAGGGGGAACGAATTATCTGGCCCCTGGGGGGCTTTCAGATTCCC
AACTTCTTCTGGAGCCTGGGGATCGGTCACACTGGTGCGTGGTGGCATACTGGGAGGAGAAGACGAGA
GTGGGGAGGCTCTACTGTGTCCAGGAGCCCTCTCTGGATATCTTCTATGATCTACCTCAGGGGAATGG
CTTTTGCCTCGGACAGCTCAATTCGGACAACAAGAGTCAGCTGGTGCAGAAGGTGCGGAGCAAAATCG
GCTGCGGCATCCAGCTGACGCGGGAGGTGGATGGTGTGTGGGTGTACAACCGCAGCAGTTACCCCATC
TTCATCAAGTCCGCCACACTGGACAACCCGGACTCCAGGACGCTGTTGGTACACAAGGTGTTCCCCGG
TTTCTCCATCAAGGCTTTCGACTACGAGAAGGCGTACAGCCTGCAGCGGCCCAATGACCACGAGTTTA
TGCAGCAGCCGTGGACGGGCTTTACCGTGCAGATCAGCTTTGTGAAGGGCTGGGGCCAGTGCTACACC
CGCCAGTTCATCAGCAGCTGCCCGTGCTGGCTAGAGGTCATCTTCAACAGCCGGTAG

We claim:

1. A codon-optimized Smad7 polynucleotide, wherein the codon-optimized Smad7 polynucleotide is set forth in SEQ ID NO: 1 or is a polynucleotide having at least 90% identity thereto.

2. (canceled)

3. The codon-optimized Smad7 polynucleotide of claim 1, wherein the polynucleotide is modified to maximize a codon adaptation index and the codon adaptation index is in a range or 0.8-1.0.

4. The codon-optimized Smad7 polynucleotide of claim 1, wherein one or more of rare tandem repeats, anti-viral motifs, hairpins and negative cis elements are eliminated.

5. (canceled)

6. The codon-optimized Smad7 polynucleotide of claim 1, wherein the codons corresponding to residues that are methylated (arginine 57 and arginine 67) are changed to code for any other amino acid other than lysine, which is optionally methylated.

7. A viral vector or a chimeric/hybrid viral vector comprising the codon-optimized Smad7 polynucleotide of claim 1.

8. (canceled)

9. (canceled)

10. (canceled)

11. The codon-optimized Smad7 polynucleotide of claim 1, wherein the codon-optimized Smad7 polynucleotide is flanked by inverted terminal repeat sequences.

12. The viral vector or a chimeric/hybrid viral vector of claim 7, wherein the viral vector or a chimeric/hybrid viral vector comprises a muscle-specific promoter, gene regulatory cassette or enhancer that directs expression of the codon-optimized polynucleotide in muscle cells.

13. (canceled)

14. The viral vector or a chimeric/hybrid viral vector of claim 7, wherein the viral vector or a chimeric/hybrid viral vector comprises a tissue-specific silencer that limits expression of the codon-optimized Smad7 polynucleotide to muscle cells or to heart cells.

15. A method of increasing or prolonging Smad7 expression in a subject, the method comprising a recombinant viral vector including a codon-optimized Smad7 polynucleotide wherein the codon-optimized Smad7 polynucleotide is modified to increase a codon adaptation index, remove rare tandem repeats and negative cis elements and/or modify a stop codon relative to a wild-type Smad7 sequence.

16. The method of claim 15, wherein the codon-optimized Smad7 polynucleotide is included in a viral vector or a chimeric/hybrid viral vector.

17. (canceled)

18. (canceled)

19. The method of claim 15, wherein the codon-optimized Smad7 polynucleotide is flanked by inverted terminal repeat sequences.

20. The method of claim 15, wherein the codon-optimized Smad7 polynucleotide is delivered to tissues using a non-viral gene delivery system.

21. The method of claim 15, wherein the codon-optimized Smad7 polynucleotide is set forth in SEQ ID NO: 1 or is a nucleotide sequence having at least 90% identity thereto

22. A method of enhancing muscle mass and/or strength in a subject, comprising administering to the subject a therapeutically effective amount of the codon-optimized Smad7 polynucleotide of claim 1 to the subject.

23. (canceled)

24. A method of treating muscle wasting in a subject diagnosed with a muscular dystrophy, comprising selecting a subject with a muscular dystrophy and administering to the subject a therapeutically effective amount of the codon-optimized Smad7 polynucleotide of claim 1.

25. A method of treating muscle wasting to increase muscle strength and/or muscle volume comprising administering the codon-optimized Smad7 polynucleotide of claim 1 to a subject.

26. A method of inhibiting or preventing muscle wasting in a subject, comprising administering to the subject a therapeutically effective amount of the codon-optimized Smad7 polynucleotide of claim 1 to the subject.

27. (canceled)

28. (canceled)

29. The method of claim 26, wherein muscle wasting is caused by microgravity stress or prolonged exposure to microgravity and/or space flight.

30. The method of claim 26, wherein the muscle wasting comprises wasting of cardiac muscle, skeletal muscle, or both.

31. The method of claim 15, comprising the delivery of the codon-optimized Smad7 polynucleotide via intramuscular or intravenous injections.

32. (canceled)

33. (canceled)
